(19) United States
(12) Patent Application Publication
(10) Pub. No.: US 2004/0224405 A1
(43) Pub. Date: Nov. 11, 2004

(54) siRNA induced systemic gene silencing in mammalian systems

(75) Inventors: Devin Leake, Denver, CO (US); Angela Reynolds, Conifer, CO (US); Anastasia Khvorova, Boulder, CO (US); William Marshall, Boulder, CO (US)

Correspondence Address: KALOW & SPRINGUT LLP, 488 MADISON AVENUE, 19TH FLOOR, NEW YORK, NY, 10022, US

(73) Assignee: Dharmacon Inc., Lafayette, CO

(21) Appl. No.: 10/431,027

(22) Filed: May 6, 2003

(51) Int. Cl. A61K 48/00; C12N 5/02

(52) U.S. Cl. 435/375; 514/44

(57) ABSTRACT

The present invention is directed to methods and compositions for performing gene silencing in mammalian cells by targeting a region of a non-protein coding target nucleic acid sequence with at least one siRNA molecule comprising a duplex region of between 19 and 30 base pairs.

### FIELD OF THE INVENTION

The present invention relates to the field of gene silencing.

### BACKGROUND

Relatively recent discoveries in the field of RNA metabolism have revealed that the uptake of double stranded RNA (dsRNA) can induce a phenomenon known as RNA interference (RNAi). RNAi is a process by which a polynucleotide directly or indirectly inhibits the activity of another nucleotide sequences, such as messenger RNA. This phenomenon has been observed in cells of a diverse group of organisms, including C. elegans, Drosophila, and humans, suggesting its promise as a powerful therapeutic approach to the genetic control of human disease.

In most organisms, RNAi is effective when using relatively long dsRNA. Unfortunately, in mammalian cells, the use of long dsRNA to induce RNAi has been met with only limited success. In large part, this ineffectiveness is due to induction of the interferon response, which results in a general, as opposed to targeted, inhibition of protein synthesis.

Recently, it has been shown that when short RNA duplexes are introduced into mammalian cells in culture, sequence-specific inhibition of target mRNA can be realized without inducing an interferon response. These short dsRNAs, referred to as small interfering RNAs (siRNAs), can, for example, act catalytically at sub-molar concentrations to cleave greater than 95% of the target mRNA in a cell. A description of the mechanisms for siRNA activity, as well as some of its applications are described in Provost et al., Ribonuclease Activity and RNA Binding of Recombinant Human Dicer, E.M.B.O.J., 2002 Nov., 1, 21(21): 5864 -5874; Tabara et al., The dsRNA Binding Protein RDE-4 Interacts with RDE-1, DCR-1 and a DexH-box Helicase to Direct RNAi in C. elegans, Cell, 2002, Jun. 28, 109(7):861-71; Ketting et al., Dicer Functions in RNA Interference and in Synthesis of Small RNA Involved in Developmental Timing in C. elegans; and Martinez et al., Single-Stranded Antisense siRNAs Guide Target RNA Cleavage in RNAi, Cell 2002, Sep. 6, 110(5):563, all of which are incorporated by reference herein.

RNA-induced gene silencing in mammalian cells is presently believed to implicate at least one of three different levels of control: (i) transcription inactivation (siRNA-guided DNA and histone modification, for example, methylation); (ii) siRNA-induced mRNA degradation; and (iii) mRNA-induced transcriptional attenuation. The interference effect by each of the actions can be long lasting and detected after many cell divisions. Transcription inactivation mediated by siRNA-guided DNA and histone modifications, such as methylation, can be particularly long lasting. Such inactivation can potentially last for the lifetime of a cell or organism. Consequently, the ability to assess gene function via siRNA mediated methods, as well as to develop therapies based on siRNA-induced gene silencing, presents an exciting and valuable tool that will accelerate genome-wide investigations across a broad range of biomedical and biological research.

The majority of the research in the area of gene silencing has focused on targeting mRNA for degradation, the second and third aforementioned activities. However, the opportunities for gene silencing by targeting non-protein coding nucleic acid sequences, and targeting siRNA to the nucleus of the cell, remain under-explored.

Gene silencing via modification of genomic DNA directed by dsRNA has been demonstrated in plants. As persons skilled in the art are aware, it has been observed in plants that long double stranded RNA indicates that it may be processed into shorter duplexes that direct methylation of DNA at many, if not all, cytosine residues within regions homologous to the dsRNA. This mechanism of silencing is mediated either directly or indirectly by DNA methyltransferases and histone acetylases and deacetylases, and presumably requires entry of the dsRNA into the cell's nucleus.

Unfortunately, successful application of this phenomenon has been limited to plants and lower organisms. Thus, there remains a need to optimize gene silencing in mammalian cells by targeting non-protein coding nucleic acid sequences and by directing siRNAs to the nucleus of a cell. The present invention offers a solution.

### SUMMARY OF THE INVENTION

The present invention is directed to compositions and methods for performing RNA interference.

According to a first embodiment, the present invention provides a method of gene silencing comprising introducing at least one siRNA molecule into a mammalian cell, wherein said at least one siRNA molecule has an antisense strand that is at least substantially complementary to a region of non-protein encoding target nucleic acid sequence and said at least one siRNA molecule comprises a duplex region of between 25 and 30 base pairs.

According to a second embodiment, the present invention provides a method of gene silencing comprising introducing into a mammalian cell at least two siRNA molecules, wherein each of said at least two siRNA molecules is comprised of a sense strand and an antisense strand, each of said antisense strands is at least substantially complementary to a region of non-protein coding nucleic acid target sequence, and within each of said at least two siRNA molecules said sense strand and said antisense strand form a duplex region of between 21 and 30 base pairs.

According to a third embodiment, the present invention provides a method of gene silencing comprising introducing at least one siRNA into a mammalian cell, wherein said at least one siRNA molecule is comprised of:

(a) a sense strand;

(b) an antisense strand that is at least substantially complementary to a region of non-protein coding target nucleic acid sequence; and

(c) a nucleus uptake modification located within at least one of said sense strand and said antisense strand.

According to a fourth embodiment, the present invention provides a method of gene silencing comprising introducing at least two siRNA molecules into a mammalian cell, wherein said at least two siRNA molecules are each comprised of:

(a) a sense strand;

(b) an antisense strand that is at least substantially complementary to a region of non-protein coding target nucleic acid sequence; and

(c) a nucleus uptake modification located within at least one of said sense strand and said antisense strand;

and the antisense strand of each of said at least two siRNA molecules is at least substantially complementary to a different region of the non-protein coding target nucleic acid sequence.

Through the use of the present invention, targeting of siRNAs to regions of non-protein coding target sequences in mammalian cells may be performed. Also, gene silencing using siRNAs targeted to regulatory sequences operably linked or operably associated with protein coding sequences in mammalian cells may be performed. The gene silencing of the present invention may be by methylation or other methods that directly or indirectly inhibit transcription or translation.

For a better understanding of the present invention together with other and further advantages and embodiments, reference is made to the following description taken in conjunction with the examples, the scope of the which is set forth in the appended claims.

### BRIEF DESCRIPTION OF THE FIGURES

The preferred embodiments of the present invention have been chosen for purposes of illustration and description but are not intended to restrict the scope of the invention in any way. The benefits of the preferred embodiments of certain aspects of the invention are shown in the accompanying figures, wherein:

FIG. 1A illustrates the effect of individual siRNAs having 19mer duplex regions directed against the CMV promoter at 24 hours post-transfection.

FIG. 1B illustrates the effect of individual siRNAs having 19mer duplex regions directed against the CMV promoter at 48 hours post-transfection.

FIG. 1C illustrates the effect of individual siRNAs having 19mer duplex regions directed against the CMV promoter at 72 hours post-transfection in human kidney HEK 293 cells.

FIG. 2A illustrates the effect of pools of siRNAs having 19mer duplex regions directed against the CMV promoter at 24 hours post-transfection in human kidney HEK 293 cells.

FIG. 2B illustrates the effect of pools of siRNAs having 19mer duplex regions directed against the CMV promoter at 48 hours post-transfection in human kidney HEK 293 cells.

FIG. 2C illustrates the effect of pools of siRNAs having 19mer duplex regions directed against the CMV promoter at 72 hours post-transfection in human kidney HEK 293 cells.

FIG. 3 illustrates the effect of cell density on silencing mediated by individual and pooled siRNAs having 19mer duplex regions and having phosphorothioate modified internucleotide linkages directed against the CMV promoter in human kidney HEK 293 cells.

FIG. 4 illustrates the effect of individual and pooled siRNAs having 19mer duplex regions, 25mer duplex regions, 27mer duplex regions, and phosphorothioate modified siRNAs having 19mer duplex regions directed against the CMV promoter at 24 hours post-transfection in human kidney HEK 293 cells.

FIG. 5 illustrates the effect of cell density on silencing mediated by individual and pooled siRNAs having 25mer duplex regions directed against the CMV promoter, at 24 hours post-transfection, in human kidney HEK 293 cells.

FIG. 6 illustrates the effect of silencing of the firefly luciferase gene mediated by individual siRNAs having 19mer duplex regions, individual siRNAs having 25mer duplex regions, individual and pooled siRNAs having 27mer duplex regions, and individual and pooled siRNAs having 19mer duplex regions and having phosphorothioate modified internucleotide linkages, directed against the CMV promoter directing firefly luciferase transcription, at 24 hours post-transfection, in human HeLa cells.

FIG. 7 illustrates the effect of silencing of the secreted human alkaline phosphatase gene mediated by individual siRNAs having 19mer duplex regions, individual siRNAs having 25mer duplex regions, individual and pooled siRNAs having 27mer duplex regions, and individual and pooled siRNAs having 19mer duplex regions and having phosphorothioate modified internucleotide linkages directed against the CMV promoter directing secreted human alkaline phosphatase transcription, at 24 hours post-transfection, in human HeLa cells.

FIG. 8 illustrates the effect of silencing of the secreted human alkaline phosphatase gene mediated by individual and pooled siRNAs directed against the CMV promoter, at 24 hours post-transfection, in human kidney HEK 293 cells.

FIG. 9 illustrates an outline of an exemplary RNA synthesis cycle.

FIG. 10 illustrates the structure of a preferred 2′-ACE protected RNA immediately prior to 2′-deprotection.

FIG. 11 depicts the DNA sequence of the promoter region of human cytomegalovirus, oriented 5′ to 3′.

FIG. 12 is a diagram of the CMV-SEAP vector pAAV6.

FIG. 13 is a diagram of the CMV-fLuc vector.

### DETAILED DESCRIPTION

Unless stated otherwise, the following terms and phrases have the meanings provided below:

### Alkyl

The term “alkyl” refers to a hydrocarbyl moiety that can be saturated or unsaturated, and substituted or unsubstituted. It may comprise moieties that are linear, branched, cyclic and/or heterocyclic, and contain functional groups such as ethers, ketones, aldehydes, carboxylates, etc.

Exemplary alkyl groups include but are not limited to substituted and unsubstituted groups of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and alkyl groups of higher number of carbons, as well as 2-methylpropyl, 2-methyl-4-ethylbutyl, 2,4-diethylpropyl, 3-propylbutyl, 2,8-dibutyldecyl, 6,6-dimethyloctyl, 6-propyl-6-butyloctyl, 2-methylbutyl, 2-methylpentyl, 3-methylpentyl, and 2-ethylhexyl. The term alkyl also encompasses alkenyl groups, such as vinyl, allyl, aralkyl and alkynyl groups.

Substitutions within an alkyl group can include any atom or group that can be tolerated in the alkyl moiety, including but not limited to halogens, sulfurs, thiols, thioethers, thioesters, amines (primary, secondary, or tertiary), amides, ethers, esters, alcohols and oxygen. The alkyl groups can by way of example also comprise modifications such as azo groups, keto groups, aldehyde groups, carboxyl groups, nitro, nitroso or nitrile groups, heterocycles such as imidazole, hydrazino or hydroxylamino groups, isocyanate or cyanate groups, and sulfur containing groups such as sulfoxide, sulfone, sulfide, and disulfide.

Further, alkyl groups may also contain hetero substitutions, which are substitutions of carbon atoms, by for example, nitrogen, oxygen or sulfur. Heterocyclic substitutions refer to alkyl rings having one or more heteroatoms. Examples of heterocyclic moieties include but are not limited to morpholino, imidazole, and pyrrolidino.

### 2′-O-alkyl modified nucleotide

The phrase “2′-O-alkyl modified nucleotide” refers to a nucleotide unit having a sugar moiety, for example a deoxyribosyl moiety that is modified at the 2′ position such that an oxygen atom is attached both to the carbon atom located at the 2′ position of the sugar and to an alkyl group.

### Amine and 2′ amine modified nucleotide

The term “amine” refers to moieties that can be derived directly or indirectly from ammonia by replacing one, two, or three hydrogen atoms by other groups, such as, for example, alkyl groups. Primary amines have the general structures RNH₂ and secondary amines have the general structure R₂NH. The phrase “2′ amine modified nucleotide” refers to a nucleotide unit having a sugar moiety that is modified with an amine or nitrogen containing group attached to the 2′ position of the sugar.

The term amine includes, but is not limited to methylamine, ethylamine, propylamine, isopropylamine, aniline, cyclohexylamine, benzylamine, polycyclic amines, heteroatom substituted aryl and alkylamines, dimethylamine, diethylamine, diisopropylamine, dibutylamine, methylpropylamine, methylhexylamine, methylcyclopropylamine, ethylcylohexylamine, methylbenzylamine, methycyclohexylmethylamine, butylcyclohexylamine, morpholine, thiomorpholine, pyrrolidine, piperidine, 2,6-dimethylpiperidine, piperazine, and heteroatom substituted alkyl or aryl secondary amines.

### Antisense Strand

The phrase “antisense strand” as used herein, refers to a polynucleotide that is substantially or 100% complementary, to a target nucleic acid of interest, such as, for example, a non-protein coding nucleic acid sequence. An antisense strand may be comprised of a polynucleotide that is RNA, DNA or chimeric RNA/DNA. For example, an antisense strand may be complementary, in whole or in part, to a non-protein coding sequence, for example, an RNA sequence that is not mRNA (e.g., tRNA, rRNA and hnRNA) or a sequence of DNA that is a non-protein coding sequence.

### Complementary

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes.

Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. Substantial complementarity refers to 79% or greater complementarity. Thus, for example, two polynucleotides of 29 nucleotide units each, wherein each comprises a di-dT at the 3′ terminus such that the duplex region spans 27 bases, and wherein 26 of the 27 bases of the duplex region on each strand are complementary, are substantially complementary since they are 96.3% complementary when excluding the di-dT overhangs. In determining complementarity, overhang regions are excluded.

### Conjugate and Terminal Conjugate

The term “conjugate” refers to a molecule or moiety that alters the physical properties of a polynucleotide such as those that increase stability and/or facilitate uptake of double stranded RNA by itself. A “terminal conjugate” may be attached directly or through a linker to a 3′ and/or 5′ end of a polynucleotide or double stranded polynucleotide. An internal conjugate may be attached directly or indirectly through a linker to a base, to the 2′ position of the ribose, or to other positions that do not interfere with Watson-Crick base pairing, for example, 5-aminoallyl uridine.

In a double stranded polynucleotide, one or both 5′ ends of the strands of polynucleotides comprising the double stranded polynucleotide can bear a conjugate, and/or one or both 3′ ends of the strands of polynucleotides comprising the double stranded polynucleotide can bear a conjugate.

Conjugates may, for example, be amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates also include steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Still other examples include thioethers such as hexyl-S-tritylthiol, thiocholesterol, acyl chains such as dodecandiol or undecyl groups, phospholipids such as di-hexadecyl-rac-glycerol, triethylammonium 1,2-di-O-hexadecyl-rac-glycero-3-H-phosphonate, polyamines, polyethylene glycol, adamantane acetic acid, palmityl moieties, octadecylamine moieties, hexylaminocarbonyl-oxycholesterol, farnesyl, geranyl and geranylgeranyl moieties.

Conjugates can also be detectable labels. For example, conjugates can be fluorophores. Conjugates may include fluorophores such as TAMRA, BODIPY, Cyanine derivatives such as Cy3 or Cy5, Dabsyl, or any other suitable fluorophore known in the art.

A conjugate may be attached to any position on the terminal nucleotide that is convenient and that does not substantially interfere with the desired activity of the polynucleotide(s) that bear it, for example the 3′ or 5′ position of a ribosyl sugar. A conjugate substantially interferes with the desired activity of an siRNA if it adversely affects its functionality such that the ability of the siRNA to mediate RNA interference is reduced by greater than 80% in an in vitro assay employing cultured cells, where the functionality is measured at 24 hours post transfection.

### Deoxynucleotide

The term “deoxynucleotide” refers to a nucleotide or polynucleotide lacking an OH group at the 2′ and/or 3′ position of a sugar moiety. Instead it has a hydrogen bonded to the 2′ and/or 3′ carbon. Within an siRNA molecule that comprises one or more deoxynucleotides, “deoxynucleotide” refers to the lack of an OH group at the 2′ position of the sugar moiety, having instead a hydrogen bonded directly to the 2′ carbon.

### Deoxyribonucleotide

The terms “deoxyribonucleotide” and “DNA” refer to a nucleotide or polynucleotide comprising at least one sugar moiety that has an H, rather than an OH, at its 2′ and/or 3′ position.

### Duplex Region

The phrase “duplex region” refers to the region in two complementary or substantially complementary polynucleotides that form base pairs with one another, either by Watson-Crick base pairing or any other manner that allows for a duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” consists of 19 base pairs. The remaining base pairs may, for example, exist as 5′ and 3′ overhangs. Further, within the duplex region, 100% complementarity is not required; substantial complementarity is allowable within a duplex region. Substantial complementarity refers to 79% or greater complementarity. For example, a mismatch in a duplex region consisting of 19 base pairs (i.e., 18 base pairs and one mismatch) results in 94.7% complementarity, rendering the duplex region substantially complementary. In another example, three mismatches in a duplex region consisting of 19 base pairs (i.e., 16 base pairs and three mismatches) results in 84.2% complementarity, rendering the duplex region substantially complementary, and so on.

### Enhancer

The term “enhancer” and phrase “enhancer sequence” refer to a variety of regulatory sequence that can increase the efficiency of transcription, without regard to the orientation of the enhancer sequence or its distance or position in space from the promoter, transcription start site, or first codon of the nucleic acid sequence encoding a protein with which the enhancer is operably linked or associated.

### Functional Concentration

The phrase or “functional concentration” refers to a concentration of siRNA that will be effective at causing a greater than or equal to 80% reduction in target sequence activity at levels of 100 nM at 24, 48, 72, and 96 hours following administration, while a “marginally functional concentration” of siRNA will be effective at causing a greater than or equal to 50% reduction of target sequence activity at 100 nM at 24 hours following administration and a “non-functional concentration” of siRNA will cause a less than 50% reduction in target sequence activity levels at 100 nM at 24 hours following administration. Target sequence activity may be measured by any method known in the art. For example, where the target sequence is a promoter, target sequence activity may be measured by level of transcription, level of the protein whose transcription is operably linked or operably associated with the promoter, or activity of the protein whose transcription is operably linked or operably associated with the promoter.

### Gene

The term “gene” as used herein includes sequences of nucleic acids that encode proteins, and sequences that do not encode proteins. For example, the term “gene” includes exons and introns. Sequences that code for proteins are, for example, sequences that are contained within exons in an open reading frame between a start codon and a stop codon. Thus, “gene” herein includes, for example, promoters, enhancers and all other sequences known in the art that control the transcription, expression, or activity of another gene, whether the other gene comprises coding sequences or non-coding sequences. In one context, for example, “gene” may be used to describe a promoter or enhancer; in another context, “gene” may be used to describe a protein-coding nucleic acid sequence. A “target gene” is a nucleic acid sequence, such as, for example, a promoter or enhancer, against which an siRNA is directed for the purpose of effectuating silencing of another gene. Either or both “gene” and “target gene” may be nucleic acid sequences naturally occurring in an organism, transgenes, viral or bacterial sequences, chromosomal or extrachromosomal, and/or transiently or chronically transfected or incorporated into the cell and/or its chromatin. Thus, for example, a “target gene” can be a promoter region, and siRNA-mediated silencing of the target gene's promoter may repress the activity of another “gene” such as a gene coding for a protein (as measured by transcription, translation, expression, or presence or activity of the gene's protein product). In another example, a “target gene” can comprise an enhancer, and siRNA mediated silencing of the enhancer may repress the functionality of an operably linked or operably associated promoter, and thus repress the activity of another “gene” such as a gene coding for a protein that is operably linked to the repressed promoter and/or enhancer.

### Gene Silencing

The phrase “gene silencing” refers to the reduction in transcription, translation or expression or activity of a nucleic acid, as measured by transcription level, mRNA level, enzymatic activity, methylation state, chromatin state or configuration, or other measure of its activity or state in a cell or biological system. “Gene silencing” refers to the reduction or amelioration of activity known to be associated with a nucleic acid sequence, such as its ability to function as a regulatory sequence, its ability to be transcribed, its ability to be translated and result in expression of a protein, regardless of the mechanism whereby such silencing occurs.

### Halogen

The term “halogen” refers to an atom of either fluorine, chlorine, bromine, iodine or astatine. The phrase “2′ halogen modified nucleotide” refers to a nucleotide unit having a sugar moiety that is modified with a halogen at the 2′ position, attached directly to the 2′ carbon.

### Histone

The term “histone” refers to a type of protein that is found in the nucleus of eukaryotic cells. The class of proteins referred to as histones are those proteins around which DNA coils in order to compact itself.

### Intemucleotide Linkage

The phrase “internucleotide linkage” refers to the type of bond or linkage that is present between two nucleotide units in a polynucleotide and may be modified or unmodified. The phrase “modified internucleotide linkage” includes all modified internucleotide linkages now known in the art or that come to be known and that, from reading this disclosure, one skilled in the art would consider useful in connection with the present invention. Internucleotide linkages may have associated counterions, and the term is meant to include such counterions and any coordination complexes that can form at the internucleotide linkages. A modified internucleotide linkage can serve as a nucleus uptake modification.

Modifications of internucleotide linkages include, but are not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that can be saturated or unsaturated and/or substituted and/or contain heteroatoms, linkages with morpholino structures, amides, polyamides wherein the bases can be attached to the aza nitrogens of the backbone directly or indirectly, and combinations of such modified internucleotide linkages within a polynucleotide. The term “thio modified internucleotide linkage” includes any internucleotide linkage that comprises at least one sulfur atom.

### Linker

A “linker” is a moiety that attaches two or more other moieties to each other such as a nucleotide and its conjugate. A linker may be distinguished from a conjugate in that while a conjugate increases the stability and/or ability of a molecule to be taken up by a cell, or imparts another attribute to the molecule, a linker merely attaches a conjugate to the molecule that is to be introduced into the cell.

By way of example, linkers can comprise modified or unmodified nucleotides, nucleosides, polymers, sugars and other carbohydrates, polyethers such as, for example, polyethylene glycols, polyalcohols, polypropylenes, propylene glycols, mixtures of ethylene and propylene glycols, polyalkylamines, polyamines such as spermidine, polyesters such as poly(ethyl acrylate), polyphosphodiesters, and alkylenes. An example of a conjugate and its linker is cholesterol-TEG-phosphoramidites, wherein the cholesterol is the conjugate and the tetraethylene glycol and phosphate serve as linkers.

### Mammalian Cell

The phrase “mammalian cell” refers to a cell of any mammal, including humans. The phrase refers to cells in vivo, such as, for example, in an organism or in an organ of an organism. The phrase also refers to cells in vitro, such as, for example, cells maintained in cell culture.

### Methylation

The term “methylation” refers to the attachment of a methyl group (—CH₃) to another molecule. Typically, when DNA undergoes methylation, a methyl group is added to a cytosine bearing nucleotide, commonly at a CpG sequence, although methylation can occur at other sites as well. Proteins, such as, for example, histone 3, may also be methylated at lysine 9.

### Non-Protein Coding Target Sequence

The phrase “non-protein coding target sequence” or “non-protein coding nucleic acid sequence” refers to a nucleic acid sequence of interest that is not contained within an exon.

### Nucleotide

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, and uracil, xanthine, inosine, and queuosine that have been modified by the replacement or addition of one or more atoms or groups. Some examples of types of modifications that can comprise nucleotides that are modified with respect to the base moieties, include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Further, the term nucleotide also includes those species that have a detectable label, such as for example a radioactive or fluorescent moiety, or mass label attached to the nucleotide.

### Nucleotide Unit

The phrase “nucleotide unit” refers to a single nucleotide residue and is comprised of a modified or unmodified nitrogenous base, a modified or unmodified sugar, and a modified or unmodified moiety that allows for linking of two nucleotides together or a nucleotide to a conjugate that precludes further linkage. The single nucleotide residue may be in a polynucleotide. Thus, a polynucleotide having 27 bases has 27 nucleotide units.

### Nucleus Uptake Modification

The phrase “nucleus uptake modification” refers to a modification to a molecule that facilitates entry into or association with a cell's nucleus. An example of a “nucleus uptake modification” is a stabilizing modification, such as a modified internucleotide linkage, that confers sufficient stability on a molecule, such as a nucleic acid, to render it resistant enough to degradation by nucleases such that it is able to accumulate in the nucleus of a cell when exogenously introduced into the cell. In this example, entry into the cell's nucleus is facilitated by the ability of the modified nucleic acid to resist nucleases sufficiently well such that an effective concentration of the nucleic acid can be achieved inside the nucleus. Alternatively, the modification allows for either passive or active uptake into the nucleus. An effective concentration is a concentration that results in a detectable change in the transcription or activity of a gene or target sequence as the result of the accumulation of nucleic acid within the nucleus.

### Operably Linked and Operably Associated

The phrases “operably associated” and “operably linked” refer to functionally related nucleic acid sequences. By way of example, a regulatory sequence is operably linked or operably associated with a protein encoding nucleic acid sequence if the regulatory sequence can exert an effect on the expression of the encoded protein. In another example, a promoter is operably linked or operably associated with a protein encoding nucleic acid sequence if the promoter controls the transcription of the encoded protein. While operably associated or operably linked nucleic acid sequences can be contiguous with the nucleic acid sequence that they control, the phrases “operably associated” and “operably linked” are not meant to be limited to those situations in which the regulatory sequences are contiguous with the nucleic acid sequences they control.

### Orthoester Protected and Orthoester Modified

The phrases “orthoester protected” and “orthoester modified” refer to modification of a sugar moiety within a nucleotide unit with an orthoester. Preferably, the sugar moiety is a ribosyl moiety. In general, orthoesters have the structure RC(OR′)₃ wherein each R′ can be the same or different, R can be an H, and wherein the underscored C is the central carbon of the orthoester. The orthoesters of the present invention are comprised of orthoesters wherein a carbon of a sugar moiety in a nucleotide unit is bonded to an oxygen, which is in turn bonded to the central carbon of the orthoester. To the central carbon of the orthoester is, in turn, bonded two oxygens, such that in total three oxygens bond to the central carbon of the orthoester. These two oxygens bonded to the central carbon (neither of which is bonded to the carbon of the sugar moiety) in turn, bond to carbon atoms that comprise two moieties that can be the same or different. For example, one of the oxygens can be bound to an ethyl moiety, and the other to an isopropyl moiety. In one example, R can be an H, one R′ can be a ribosyl moiety, and the other two R′ moieties can be 2-ethyl-hydroxyl moieties. Orthoesters can be placed at any position on the sugar moiety, such as, for example, on the 2′, 3′ and/or 5′ positions. Preferred orthoesters, and methods of making orthoester protected polynucleotides, are described in U.S. Pat. Nos. 5,889,136 and 6,008,400, each herein incorporated by reference in its entirety.

### Overhang

The term “overhang” refers to terminal non-base pairing nucleotides resulting from one strand extending beyond the other strand within a doubled stranded polynucleotide. One or both of two polynucleotides that are capable of forming a duplex through hydrogen bonding of base pairs may have a 5′ and/or 3′ end that extends beyond the 3′ and/or 5′ end of complementarity shared by the two polynucleotides. The single-stranded region extending beyond the 3′ and/or 5′ end of the duplex is referred to as an overhang.

### Pharmaceutically Acceptable Carrier

The phrase “pharmaceutically acceptable carrier” refers to compositions that facilitate the introduction of dsRNA into a cell and includes but is not limited to solvents or dispersants, coatings, anti-infective agents, isotonic agents, agents that mediate absorption time or release of the inventive polynucleotides and double stranded polynucleotides. Examples of “pharmaceutically acceptable carriers” include liposomes that can be neutral or cationic, can also comprise molecules such as chloroquine and 1,2-dioleoyl-sn-glycero-3-phosphatidylethanolamine, which can help destabilize endosomes and thereby aid in delivery of liposome contents into a cell, including a cell's nucleus. Examples of other pharmaceutically acceptable carriers include poly-L-lysine, polyalkylcyanoacrylate nanoparticles, polyethyleneimines, and any suitable PAMAM dendrimers (polyamidoamine) known in the art with various cores such as, for example, ethylenediamine cores, and various surface functional groups such as, for example, cationic and anionic functional groups, amines, ethanolamines, aminodecyl.

### Polynucleotide

The term “polynucleotide” refers to polymers of nucleotides, and includes but is not limited to DNA, RNA, DNA/RNA hybrids including polynucleotide chains of regularly and irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides wherein the attachment of various entities or moieties to the nucleotide units at any position are included.

### Polyribonucleotide

The term “polyribonucleotide” refers to a polynucleotide comprising two or more modified or unmodified ribonucleotides and/or their analogs.

### Promoter

The term “promoter” refers to a nucleic acid sequence that does not code for a protein, and that is operably linked or operably associated to a protein coding or RNA coding nucleic acid sequence such that the transcription of the operably linked or operably associated protein coding or RNA coding nucleic acid sequence is controlled by the promoter. Typically, eukaryotic promoters comprise between 100 and 5,000 base pairs, although this length range is not meant to be limiting with respect to the term “promoter” as used herein. Although typically found 5′ to the protein coding nucleic acid sequence to which they are operably linked or operably associated, promoters can be found in intron sequences as well. Anecdotal evidence suggests that, in certain cases, promoters can be found within exons, for example, in certain sequences wherein sense and antisense strands each encode proteins.

The term “promoter” is meant to include regulatory sequences operably linked or operably associated with the same protein or RNA encoding sequence that is operably linked or operably associated with the promoter. Promoters can comprise many elements, including regulatory elements.

Regulatory elements are nucleic acid sequences that regulate, induce, repress, or otherwise mediate the transcription, translation of a protein or RNA coded by a nucleic acid sequence with which they are operably linked or operably associated. Typically, a regulatory element or sequence such as, for example, an enhancer or repressor sequence, is operatively linked or operatively associated with a protein or RNA coding nucleic acid sequence if the regulatory element or regulatory sequence mediates the level of transcription, translation or expression of the protein coding nucleic acid sequence in response to the presence or absence of one or more regulatory factors that control transcription, translation and/or expression. Regulatory factors include, for example, transcription factors. Regulatory sequences may be found in introns. Regulatory sequences or element include, for example, “TATAA” boxes, “CAAT” boxes, differentiation-specific elements, cAMP binding protein response elements, sterol regulatory elements, serum response elements, glucocorticoid response elements, transcription factor binding elements such as, for example, SPI binding elements, and the like. A “CAAT” box is typically located upstream (in the 5′ direction) from the start codon of a eukaryotic nucleic acid sequence encoding a protein or RNA. Examples of other regulatory sequences include splicing signals, polyadenylation signals, termination signals, and the like. Further examples of nucleic acid sequences that comprise regulatory sequences include the long terminal repeats of the Rous sarcoma virus and other retroviruses. An example of a regulatory sequence that controls tissue-specific transcription is the interferon-epsilon regulatory sequence that preferentially induces production of the operably linked sequence encoding the protein in placental, tracheal, and uterine tissues, as opposed to lung, brain, liver, kidney, spleen, thymus, prostate, testis, ovary, small intestine, and pancreatic tissues. Many, many regulatory sequences are known in the art, and the foregoing is merely illustrative of a few.

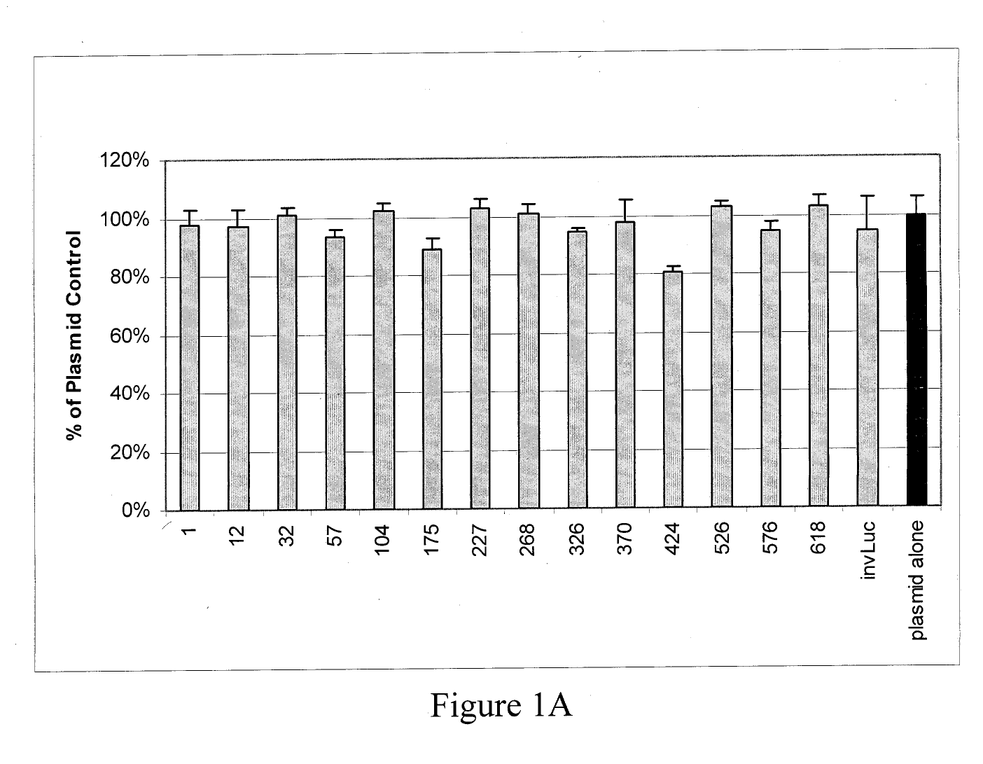

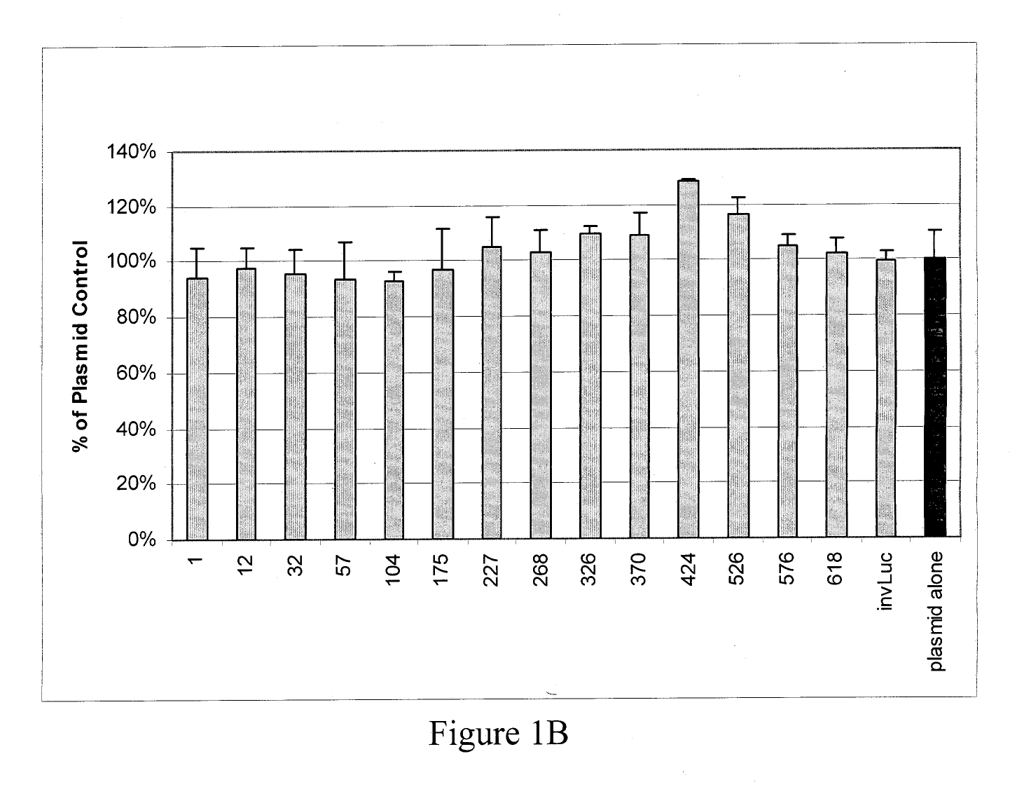

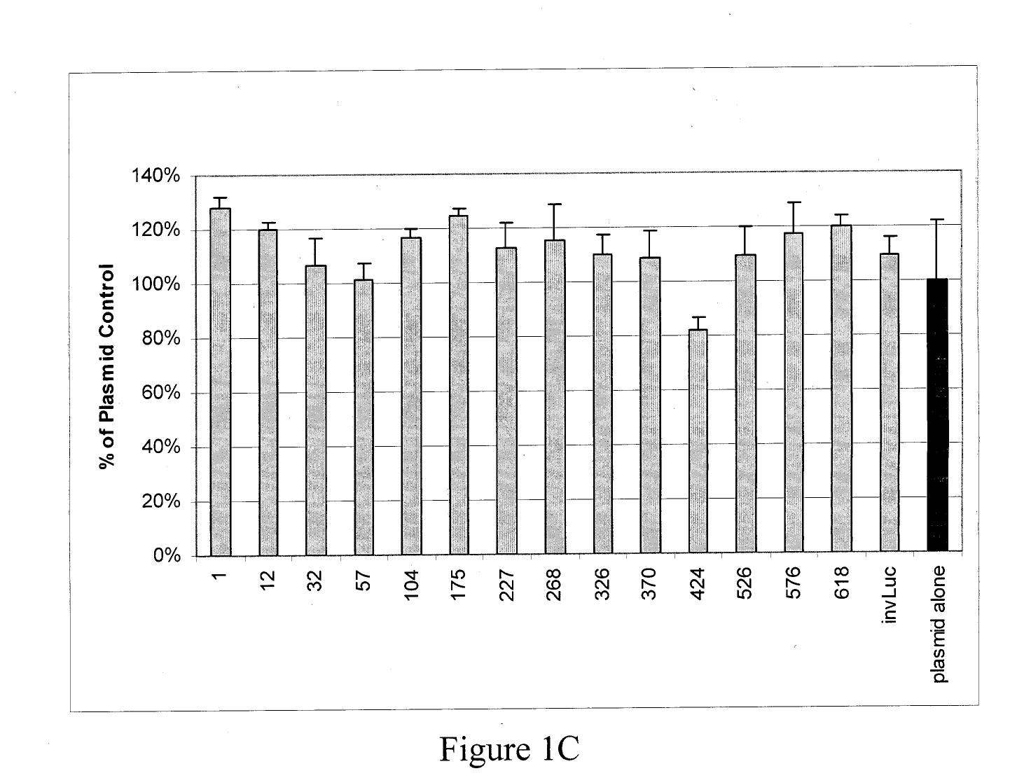

The term “promoter” comprises promoters that are inducible, wherein the transcription of the operably linked nucleic acid sequence encoding the protein is increased in response to an inducing agent. The term “promoter” may also comprise promoters that are constitutive, or not regulated by an inducing agent. An example of the sequence of a promoter, the human cytomegalovirus (CMV) promoter, is provided in FIG. 1.

### Ribonucleotide and Ribonucleic Acid

The term “ribonucleotide” and the phrase “ribonucleic acid” (RNA), refer to a modified or unmodified nucleotide or polynucleotide comprising at least one ribonucleotide unit. A ribonucleotide unit comprises an oxygen attached to the 2′ position of a ribosyl moiety that has a nitrogenous base attached in N-glycosidic linkage at the 1′ position of a ribosyl moiety, and a moiety that either allows for linkage to another nucleotide or precludes linkage.

### RNA Interference and RNAi

The phrase “RNA interference” and the term “RNAi” refer to the process by which a polynucleotide or double stranded polynucleotide comprising at least one ribonucleotide unit exerts an effect on a biological process. The process includes but is not limited to gene silencing by degrading mRNA, interactions with tRNA, rRNA, hnRNA, cDNA and genomic DNA, as well as methylation of DNA and ancillary proteins.

### Sense Strand

The phrase “sense strand” refers to a polynucleotide that has the same nucleotide sequence, in whole or in part, as a target nucleic acid such as a messenger RNA or a sequence of DNA.

### siRNA or Short Interfering RNA

The term “siRNA” and the phrase “short interfering RNA” refer to a double stranded nucleic acid that is capable of performing RNAi and that is between 18 and 30 base pairs in length (i.e., a duplex region of between 18 and 30 base pairs). Additionally, the term siRNA and the phrase “short interfering RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides.

siRNAs can be duplexes, and can also comprise short hairpin RNAs, RNAs with loops as long as, for example, 4 to 23 or more nucleotides, RNAs with stem loop bulges, micro-RNAs, and short temporal RNAs. RNAs having loops or hairpin loops can include structures where the loops are connected to the stem by linkers such as flexible linkers. Flexible linkers can be comprised of a wide variety of chemical structures, as long as they are of sufficient length and materials to enable effective intramolecular hybridization of the stem elements. Typically, the length to be spanned is at least about 10-24 atoms.

### Stabilized

The term “stabilized” refers to the ability of a dsRNA to resist degradation while maintaining functionality and can be measured in terms of its half-life in the presence of, for example, biological materials such as serum. The half-life of an siRNA in, for example, serum refers to the time taken for the 50% of siRNA to be degraded.

Wherever a range of values is provided in this disclosure, each intervening value, unless the context dictates otherwise, is encompassed within the invention. Further, it is understood that the invention includes, for each value, tenths of the lower limit indicated, unless the context clearly dictates otherwise. The invention also includes the upper and lower limit of the stated range, unless otherwise indicated. The upper and lower limits of smaller ranges may independently be included in the smaller ranges. Where the stated range includes one or both of the limits, ranges excluding either or both of those included limits are also included in the invention.

Wherever an inconsistency exists as to the meaning of a term used herein, between material incorporated by reference and this disclosure, the definitions and terms of this disclosure is controlling as to the meaning of the term.

### PREFERRED EMBODIMENTS

The present invention will now be described in connection with preferred embodiments. These embodiments are presented to aid in an understanding of the present invention and are not intended, and should not be construed, to limit the invention in any way. All alternatives, modifications and equivalents that may become apparent to those of ordinary skill upon reading this disclosure are included within the spirit and scope of the present invention.

This disclosure is not a primer on compositions and methods for performing RNA interference. Basic concepts known to those skilled in the art have not been set forth in detail.

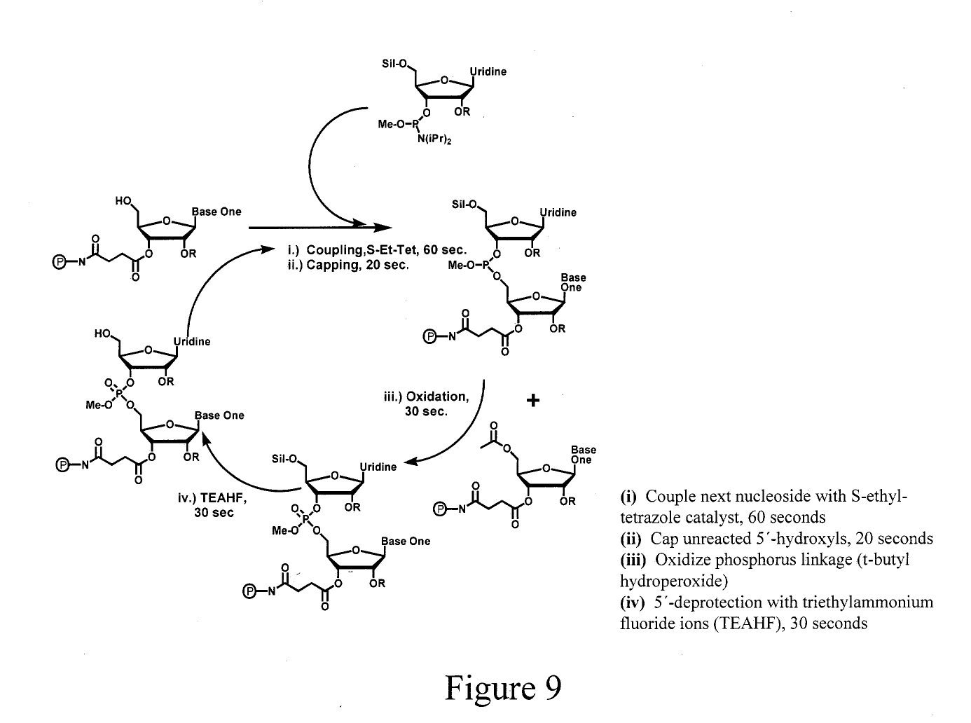

According to a first embodiment, the present invention provides a method of gene silencing comprising introducing at least one siRNA molecule into a mammalian cell's nucleus, wherein said at least one siRNA molecule has an antisense strand that is at least substantially complementary to a region of a non-protein coding target nucleic acid sequence and said at least one siRNA molecule comprises a duplex region of between 25 and 30 base pairs. The duplex region of the siRNA molecule preferably comprises between 26 and 29 base pairs. Preferably, the antisense strand is substantially complementary to the region of non-protein coding target nucleic acid sequence. More preferably, the antisense strand is 100% complementary to the region of non-protein coding target nucleic acid sequence. The sense strand is preferably substantially complementary to the region of the antisense strand with which it forms a duplex (excluding overhangs, if present). More preferably, the sense strand is 100% complementary to the region of the antisense strand with which it forms a duplex. The antisense strand and/or sense strand may have overhang regions of any length. If they have overhang regions, these regions are preferably 6 nucleotides or fewer in length, more preferably 3 nucleotides or fewer in length and most preferably two nucleotides in length. The nucleotides of the at least one siRNA, or at least one strand of a duplex siRNA, may be modified or unmodified. A preferred modification is a 2′-O-bis(2-hydroxyethoxy)methyl orthoester, illustrated in FIG. 9.

Preferably, the non-coding target nucleic acid sequence comprises a promoter. The promoter can comprise one or more regulatory sequences.

After entering the mammalian cell, the at least one siRNA molecule will preferably cause modification of at least one molecule in the cell. Preferably, the at least one molecule is DNA or a histone. Preferably, the modification is the placement of one or more methyl groups on the DNA molecule or histone.

The siRNA may also contain stabilization modifications such as orthoesters, 2′-O-methyl groups, fluoro groups and stabilizing conjugates as described in commonly assigned co-pending application entitled Stabilized Polynucleotides for use in RNA Interference, filed Apr. 2, 3003, the entire disclosure of which is herein incorporated by reference.

The siRNA may be synthesized by any method that is now known or that comes to be known for synthesizing siRNA molecules and that from reading this disclosure, one skilled in the art would conclude would be useful in connection with the present invention. For example, one may use methods of chemical synthesis such as methods that employ Dharmacon, Inc.'s proprietary ACE® technology. Alternatively, one could also use template dependant synthesis methods.

The siRNA may be introduced to the nucleus of a cell by any method that is now known or that comes to be known and that from reading this disclosure, persons skilled in the art would determine would be useful in connection with the present invention in enabling siRNA to cross the cellular membrane and the nuclear membrane. These methods include, but are not limited to, any manner of transfection, such as for example transfection employing DEAE-Dextran, calcium phosphate, cationic lipids/liposomes, micelles, manipulation of pressure, microinjection, electroporation, immunoporation, use of vectors such as viruses, plasmids, cosmids, bacteriophages, cell fusions, and coupling of the polynucleotides to specific conjugates or ligands such as antibodies, antigens, or receptors, passive introduction, adding moieties to the siRNA that facilitate its uptake, and the like.

According to a second embodiment, the present invention provides a method of gene silencing comprising introducing into a mammalian cell at least two siRNA molecules, wherein each of said at least two siRNA molecules is comprised of a sense strand and an antisense strand, each of said antisense strands is at least substantially complementary to a region of non-protein coding nucleic acid target sequence, and within each of said at least two siRNA molecules said sense strand and said antisense strand form a duplex region of between 19 and 30 base pairs. The duplex region of the siRNA molecule preferably comprises between 26 and 29 base pairs. Preferably, the antisense strand of each of the at least two siRNA molecules is at least substantially complementary to a region of the same non-protein coding target nucleic acid sequence. More preferably, the antisense strand of each siRNA is 100% complementary to the region of non-protein coding target nucleic acid sequence. The sense strand of each siRNA is preferably substantially complementary to the region of the antisense strand with which it forms a duplex (excluding overhangs, if present). More preferably, the sense strand of each siRNA is 100% complementary to the region of the antisense strand with which it forms a duplex. The antisense strand and/or sense strand of each siRNA may have overhang regions of any length. If they have overhand regions, these regions are preferably 6 nucleotides or fewer in length, more preferably 3 nucleotides or fewer in length and most preferably two nucleotides in length. The nucleotides of the one or more of the at least two siRNAs, on one or more siRNAs, on either or both strands of each siRNA, may be modified or unmodified.

Preferably, the non-protein coding target nucleic acid sequence comprises a promoter. The promoter can comprise one or more regulatory sequences. Preferably, the antisense strands of at least two siRNA molecules are at least substantially complementary in their duplex region to non-overlapping sequences of said non-protein coding target nucleic acid sequence. More preferably, the antisense strands are 100% complementary in their duplex region to non-overlapping sequences of said non-protein coding target nucleic acid sequences.

After entering the mammalian cell, one or more of the siRNA molecules will preferably cause modification of at least one molecule in the cell. Preferably, the modification is methylation. Preferably, the at least one molecule is DNA or a histone.

The siRNA may also contain stabilization modifications as described in connection with the first embodiment. Further, the siRNAs may be synthesized in the same manner as described in connection with the first embodiment, and the siRNAs may be introduced into the mammalian cell in the same manner described in connection with the first embodiment.

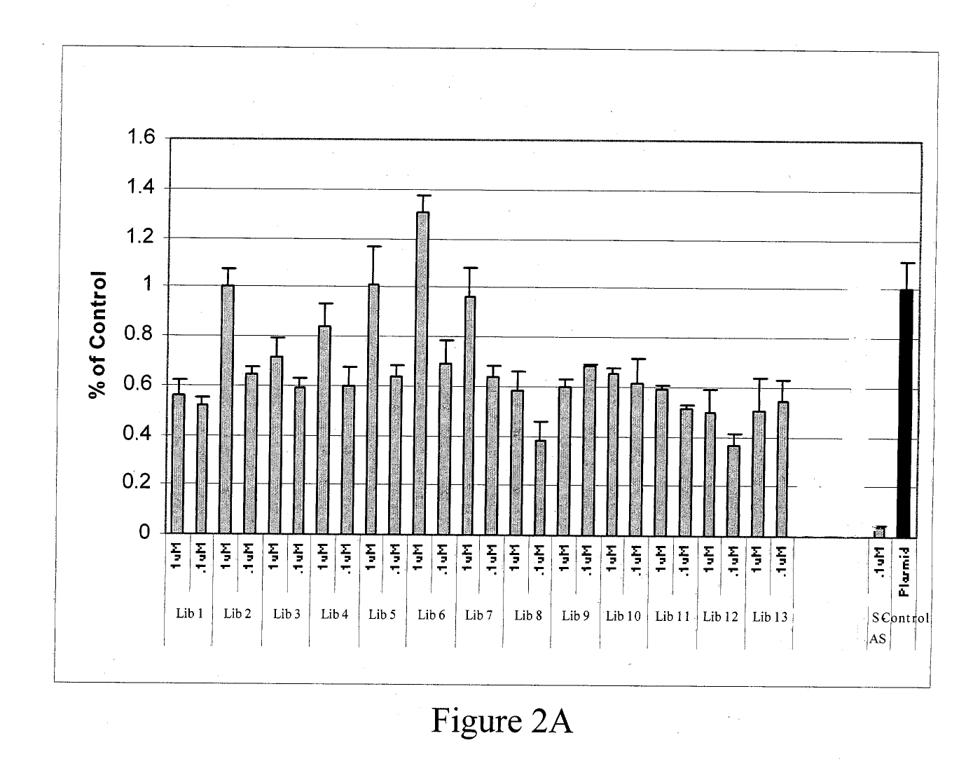

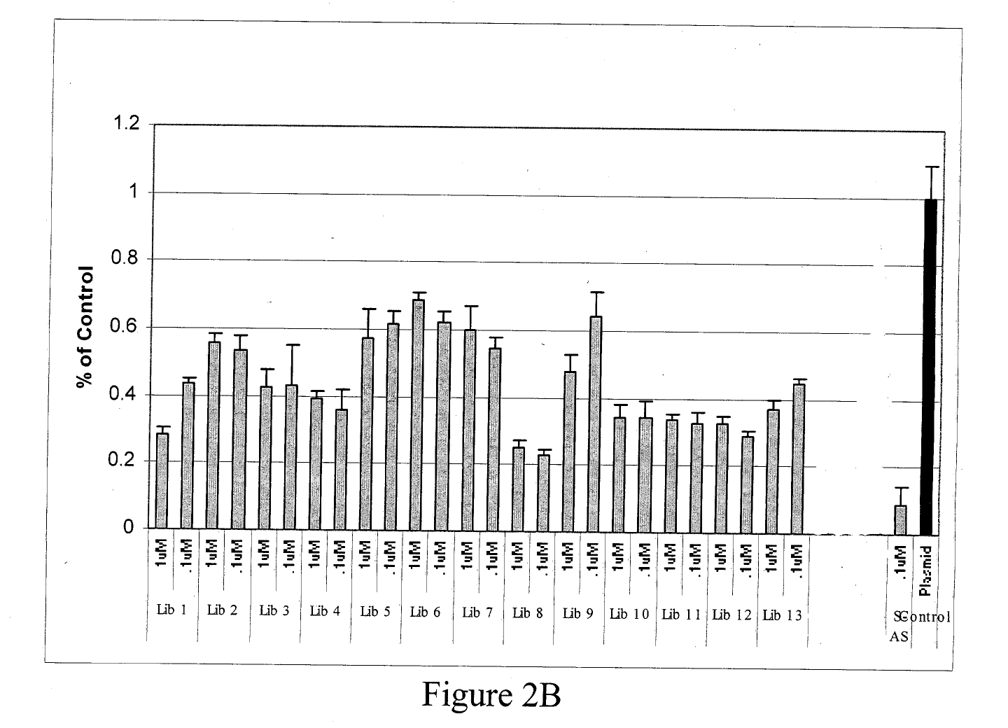

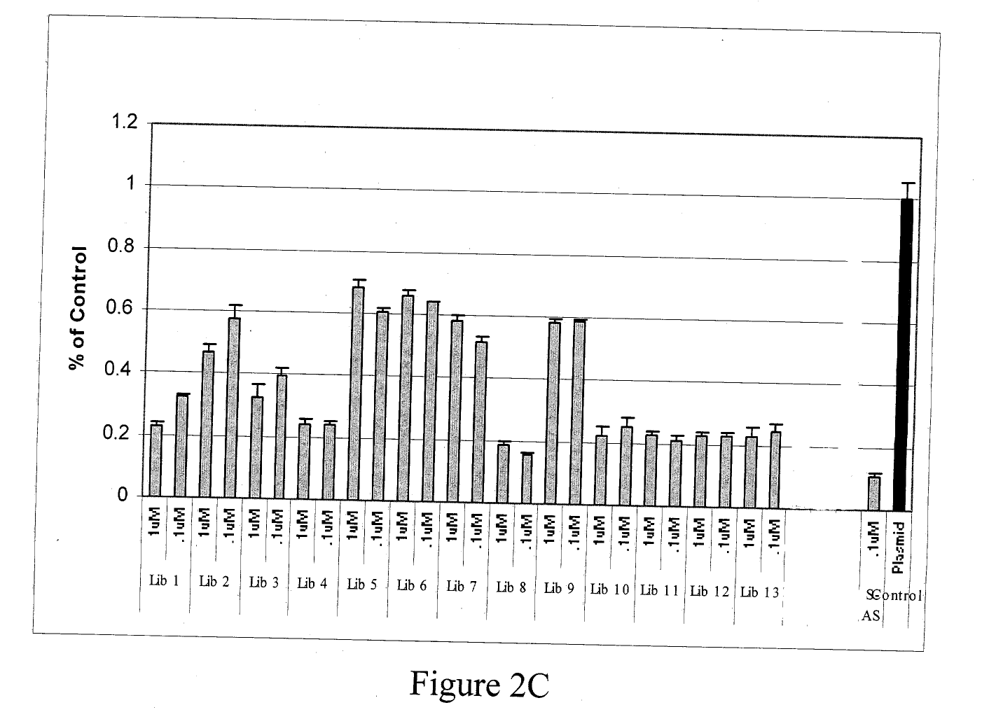

Certain fundamental advantages of the present invention, including the first and second embodiments, as well as embodiments described below, can be understood with reference to FIGS. 1A through 1C and 2A through 2C.

FIGS. 1A through 1C illustrate the effects of 21mer siRNAs having 19mer duplex regions (i.e., with 5′ and 3′ di-dT overhangs) directed against certain regions of the cytomegalovirus (CMV) promoter, using a vector construct wherein the CMV promoter drives transcription of secreted human alkaline phosphatase (SEAP). This vector, having CMV promoter-driven SEAP, was transfected into human kidney HEK 293 cells. The human alkaline phosphatase is secreted into the culture medium when the SEAP gene is transcribed and expressed. Thus, CMV promoter-driven transcription of the SEAP gene can be measured by observing the activity of the SEAP protein in the culture medium. Co-transfection of this vector with individual 21mer siRNAs having 19mer duplex regions directed against CMV promoter regions (see Table 1 for sequences) did not result in any appreciable silencing of the CMV promoter, as reflected in reporter gene activity in the culture medium over the course of 72 hours (see FIGS. 1A-1C). Thus, a variety of individual 21mer siRNAs having 19mer duplex regions, homologous to and directed against regions of the CMV promoter, fail to silence transcription directed by the promoter.

However, when multiple 21mer siRNA having 19mer duplex regions are directed against the CMV promoter are pooled and co-transfected, silencing of the promoter is observed (see FIGS. 2A-2C). FIG. 2A-2C illustrates CMV promoter silencing as the result of co-transfecting pools of siRNAs having 19mer duplex regions into human kidney HEK 293 cells, including the use of siRNAs that fail to silence when transfected individually. For example, the pool of siRNAs indicated as Library 8 in FIGS. 2A-2C include siRNAs designated 326, 370, 424 and 526 (see Tables 1 and 2). By reference to FIG. 1A-1C, these siRNAs, when transfected individually into the same cell line having the same CMV-driven reporter, fail to silence the promoter at all. However, when co-transfected together, they result in significant promoter silencing by 24 hours post-transfection (FIG. 2A), which reaches about 70% silencing by 48 hours (FIG. 2B), and at least about 80% silencing within 72 hours. With reference to FIGS. 2A-C, it is apparent that some pools silence more effectively than others. Thus, 21mer siRNAs having 19mer duplex regions, directed against a promoter region, can result in significant silencing of the promoter when pools of such siRNAs are used, but not when individual siRNAs that are 21mers having 19mer duplex regions are used separately.

According to a third embodiment, the present invention provides a method of gene silencing comprising introducing at least one siRNA into a mammalian cell, wherein said at least one siRNA molecule is comprised of a sense strand, an antisense strand that is at least substantially complementary to a region of non-protein coding target nucleic acid sequence, and a nucleus uptake modification located within at least one of said sense strand and said antisense strand.

Preferably, the nucleus uptake modification is comprised of at least one thio modified internucleotide linkage, and the modification is a phosphorothioate modification. More preferably, the nucleus uptake modification is comprised of at least four consecutive thio modified internucleotide linkages, and the modified linkages are phosphorothioate modifications. More preferably, the nucleus uptake modification is comprised of at least four consecutive thio modified internucleotide linkages, wherein the linkages are located at a 5′ terminus or 3′ terminus of at least one strand of said siRNA molecule and within a duplex region, and the linkages are phosphorothioate modifications. Most preferably, all internucleotide linkages are phosphorothioate modifications.

When there are consecutive thio linkages, the linkages occur between consecutive bases. Thus, “four consecutive thio modified internucleotide linkages” refers to the presence of a sulfur moiety between each two consecutive nucleotides within a stretch of five consecutive nucleotides.

Preferably, the antisense strand of each of the at least one siRNA molecule is at 100% complementary to the region of a non-protein coding target nucleic acid sequence. The sense strand of the at least one siRNA is preferably substantially complementary to the region of the antisense strand with which it forms a duplex (excluding overhang regions, if present). More preferably, the sense strand of the at least one siRNA is 100% complementary to the region of the antisense strand with which it forms a duplex. The antisense strand and/or sense strand of may have overhang regions of any length. If one or more overhang regions are present, these regions are preferably 6 nucleotides or fewer in length, more preferably 3 nucleotides or fewer in length and most preferably two nucleotides in length. The nucleotides of at least one siRNA, or at least one strand of a duplex siRNA, may be modified or unmodified. Preferably, the non-protein coding target nucleic acid sequence comprises a promoter. The promoter may comprise one or more regulatory sequences. The duplex region of the siRNA molecule preferably comprises between 19 and 29 base pairs. More preferably, between 26 and 29 base pairs.

After entering the mammalian cell, one or more of the siRNA molecules will preferably cause methylation of at least one molecule in the cell. Preferably, the at least one molecule DNA or a histone. Preferably, the modification is a the placement of one or more methyl groups on the DNA molecule or histone.

As in the first and second embodiments, the siRNA may also contain stabilization modifications, the siRNAs may be synthesized, and the siRNAs may be introduced into the mammalian cell in the same manner, as described in connection with the previous embodiments.

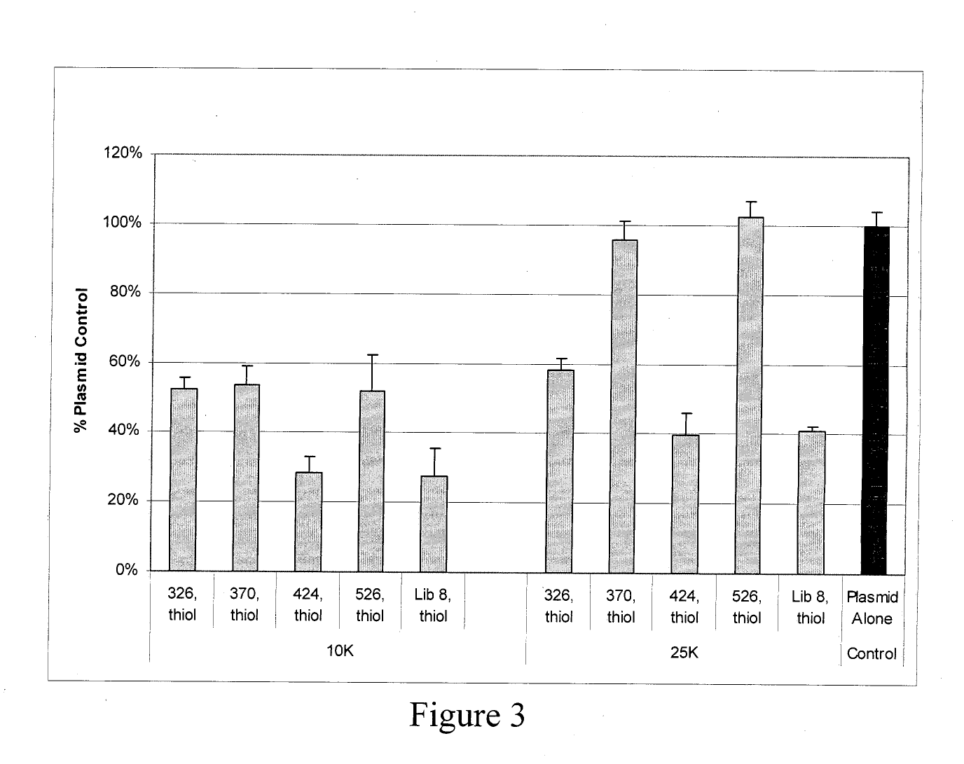

The advantages of using an siRNA with a nucleus uptake modification is demonstrated in FIG. 3. FIG. 3 is instructive in two aspects: the benefit of using nucleus uptake modifications, and the benefit of selecting an appropriate stage in cell growth for optimizing promoter silencing by siRNAs. In this experiment, individual 21mer siRNAs having 19mer duplex regions (i.e., each having a 5′ and 3′ di-dT overhang) having a nucleus uptake modification were transfected individually into human kidney HEK 293 cells. The nucleus uptake modification in each case was a modification of each internucleotide linkage with a phosphorothioate linkage.

In contrast to the lack of silencing observed using siRNAs that lack nucleus uptake modifications directed against CMV promoter regions (see FIGS. 1A-1C), silencing of the CMV promoter is observed when individual 21mer siRNAs having 19mer duplex regions and having phosphorothioate modified internucleotide linkages are transfected individually.

Without wishing to be bound by any particular theory, this striking observation may reflect that the nuclease-resistance capacity of phosphorothioate modified siRNAs allow these modified siRNAs to enter the nucleus and thus initiate or to promote the use of the cell's chromatin modifying machinery, including but not limited to its chromatin methylation/demethylation and acetylation/deacetylation activity, to silence directly or indirectly the CMV promoter of the transfected vector. Unmodified siRNAs might not be able to accumulate in the nucleus, and in fact might be turned over by intracellular nucleases at a rate that precludes the formation of any appreciable effective concentration of the siRNA in the cell's nucleus. The degree of nuclease resistance conferred by the phosphorothioate modifications may determine the ability to silence non-coding control elements such as promoters, due to the accumulation of an effective concentration of the siRNA in the cell's nucleus.

Regardless of whether the silencing of the CMV promoter is effected through direct methylation of the CMV promoter, or through some other means—with or without utilizing the cell's chromatin modifying activity—the end result is that modifications that confer stability and/or nuclease resistance, such as, for example, phosphorothioate modified internucleotide linkages, can render an otherwise nonfunctional siRNA (see FIG. 1A-C) into a functional siRNA (see FIG. 3), for the purpose of silencing a non-protein coding sequence such as a promoter. It will be apparent that there are a great many modifications known in the art that can be made to siRNAs to confer some resistance to nuclease degradation. Examples of such modifications are disclosed in the U.S. patent application “Stabilized Polynucleotides for Use in RNA Interference,” filed on Apr. 2, 2003 (Leake, et al., Ser. No. to be assigned), incorporated herein by reference in its entirety. Any modification that is now known, comes to be known, or is arrived at from reading the present disclosure, and would be useful in the present invention, may be used. Preferred modified internucleotide linkages are as stated above.

In a second aspect, FIG. 3 illustrates the effect of state of cell growth on silencing by siRNAs directed against non-coding control elements such as promoters. The experiment in FIG. 3 was conducted under two sets of growth conditions: in the first set, the human kidney HEK 293 cells were plated in 96 well plates at a density of 10,000 cells/well (about 10-20% confluency); in the second set, the human kidney HEK 293 cells were plated in 96 well plates at a density of 25,000 cells/well (about 70-80% confluency). Transfections were done with the indicated phosphorothioate modified 19mers 12 hours after plating (where 10,000 cells/well results in about 70-80% confluency, and where 25,000 cells/well results in about complete confluency), and reporter gene activity was measured 24 hours after transfection. For each individual siRNA, and on average, more effective silencing by siRNAs directed against the CMV promoter was observed in the wells plated at lower cell density. The sequences of the siRNAs used in the experiment illustrated in FIG. 3 are listed in Table 3. Library 8 represents a pool of he 21mer siRNAs having 19mer duplex regions, designated 326, 370, 424, and 526, each having all internucleotide linkages as phosphorothioate modifications (except the terminal di-dTs).

Without wishing to be bound by any particular theory, these observations are consistent with a methylation-dependent mechanism of promoter silencing. When cells are plated at low density, they actively divide until they achieve a state of confluence. Once confluent, the cells are relatively quiescent. While dividing, the genomes of the cells are actively replicating. During the genome replication process, the cell's chromatin modification machinery, including DNA and histone methylases and histone acetylases and deacetylates, are actively modifying the cell's chromatin by methylation/demethylation and acetylation/deacetylation the chromatin so that the daughter cell's chromatin acquires a distinct methylation and acetylation pattern, including both DNA and histone modifications. These modification patterns can have a profound effect on the transcriptional functionality of certain areas of the genome, and are believed to be responsible for many heritable phenotypes due to, for example, allele silencing observed as the result of imprinting. Thus, in actively replicating cells, the chromatin modifying machinery is actively working, and promoter silencing can thus be observed more readily due to the presence of factors required for methylation-dependent silencing, regardless of whether direct methylation of the promoter region is responsible for the promoter silencing effect. Thus, in in vitro applications, preferably cells are treated with siRNAs at a growth stage in which they are less than 100% confluent. More preferably, cells are treated with siRNAs at a growth stage in which they are less than about 90% confluent. Most preferably, cells are treated with siRNAs at a growth stage in which they are about 70-80% confluent. Generally, the lower the cell density, the lower the ratio between lipid and RNA required. The preferred degree of confluency may vary according to cell type. Optimization of transfection protocols are well known in the art. For example, cells can be plated at varying densities, and mixtures of varying content (varying transfection agents, such as lipids, and RNA concentrations) can be added to the cells, and functionality and toxicity levels measured without undue experimentation. As is known in the art, such standard optimization measures can be carried out for any cell line. The methods and compositions of the present invention may be particularly useful in gene silencing in rapidly dividing cells, such as cancer cells.

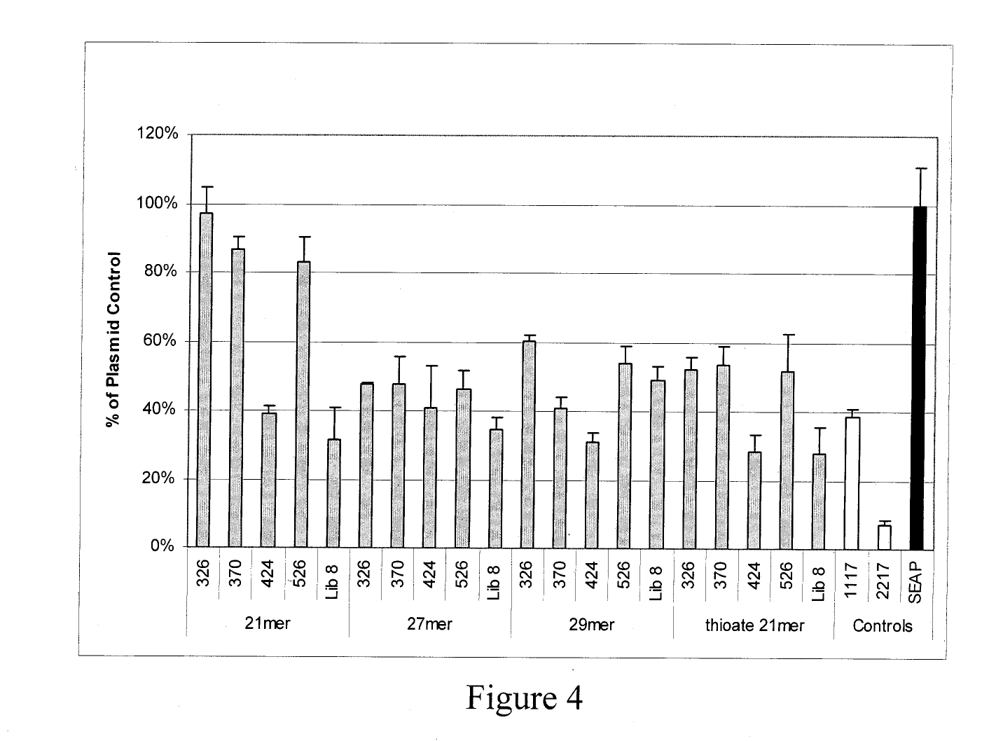

Yet another surprising aspect of siRNA silencing of non-coding control elements such as promoters is that whereas naked, or unmodified, 21mers having 19mer duplex regions are relatively ineffective in silencing a promoter, longer naked siRNAs can, when transfected individually, result in a significant degree of silencing (see FIG. 4). In FIG. 4, human kidney HEK 293 cells were transfected with individual and pooled unmodified, or naked, 21mer siRNAs having 19mer duplex regions, 27mers having 25mer duplex regions, 29mers having 27mer duplex regions, or 21mer phosphorothioate modified siRNAs having 19mer duplex regions directed against regions of the CMV promoter. Twenty four hours following transfection, SEAP reporter gene activity was measured. The naked 21mers having 19mer duplex regions, as expected (see FIG. 1A-C), on average did not result in significant promoter silencing. Surprisingly, however, the 27mers having 25mer duplex regions and the 29mers having 27mer duplex regions, individually or in pools, silenced the promoter by about 40 to about 70% (see FIG. 4). As expected, phosphorothioate modified 21mers having 19mer duplex regions also were able to silence the promoter by about over 40 to about 70% (see FIG. 4). The sequences of the siRNAs used in the experiment illustrated in FIG. 4 are listed in Table 4. Thus, siRNA-mediated silencing of non-coding control elements such as promoters is preferably performed with siRNAs greater than or equal to 19mer duplex regions. Preferably, siRNA-mediated silencing of non-coding control elements such as promoters is performed with siRNAs that have duplex regions of 19 to 30 nucleotides. More preferably, the siRNAs have duplex regions of 25 to 30 nucleotides. Most preferably, siRNA-mediated silencing of non-coding control elements such as promoters is performed with siRNAs that have duplex regions of 26 to 29 nucleotides. Although longer siRNA duplexes might be as effective or better, they are undesirable-at least for therapeutic uses-because longer siRNAs are known to induce nonspecific inflammatory responses in mammals, such as interferon-mediated responses, which are detrimental to the organism and/or cells of the organism.

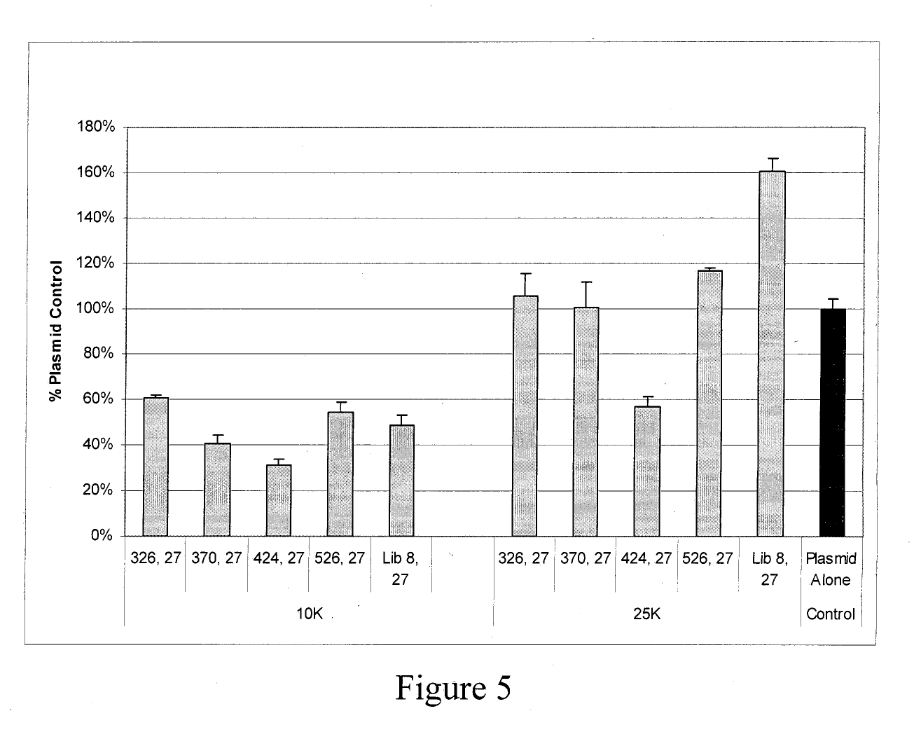

Dependence of promoter silencing on cell growth holds true for siRNAs that have duplex regions longer than 19mer duplex regions (see FIG. 4). Individually transfecting human kidney HEK 293 cells with 27mer siRNAs having duplex regions of 25 nucleotide units targeted against the CMV promoter is more effective when cells are transfected at about 70-80% confluence, as shown in FIG. 5. Individually, and on average, the 27mers with 25mer duplex regions are more effective when transfected into non-confluent cells (plated at 10,000 cells/well in a 96 well plate, then transfected 12 hours later at 70-80% confluence) than into confluent cells (plated at 25,000 cells/well, then transfected 12 hours later at confluency). At sub-confluency, silencing up to 70% can be achieved, whereas at confluency, silencing up to only about 40% is observed (see FIG. 5). The sequences of the siRNAs used in the experiment illustrated in FIG. 5 are listed in Table 5.

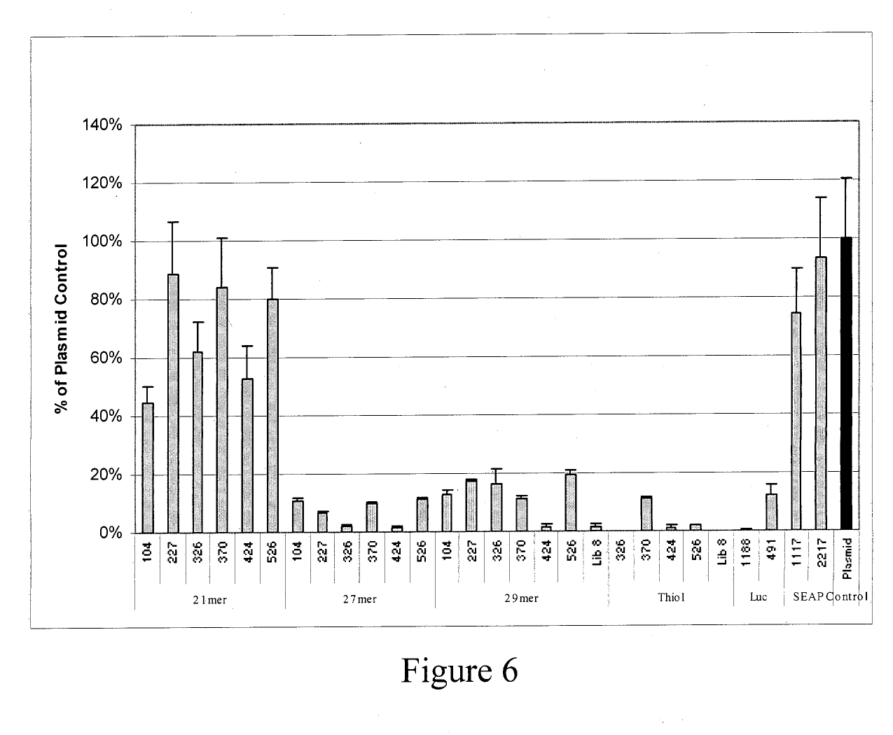

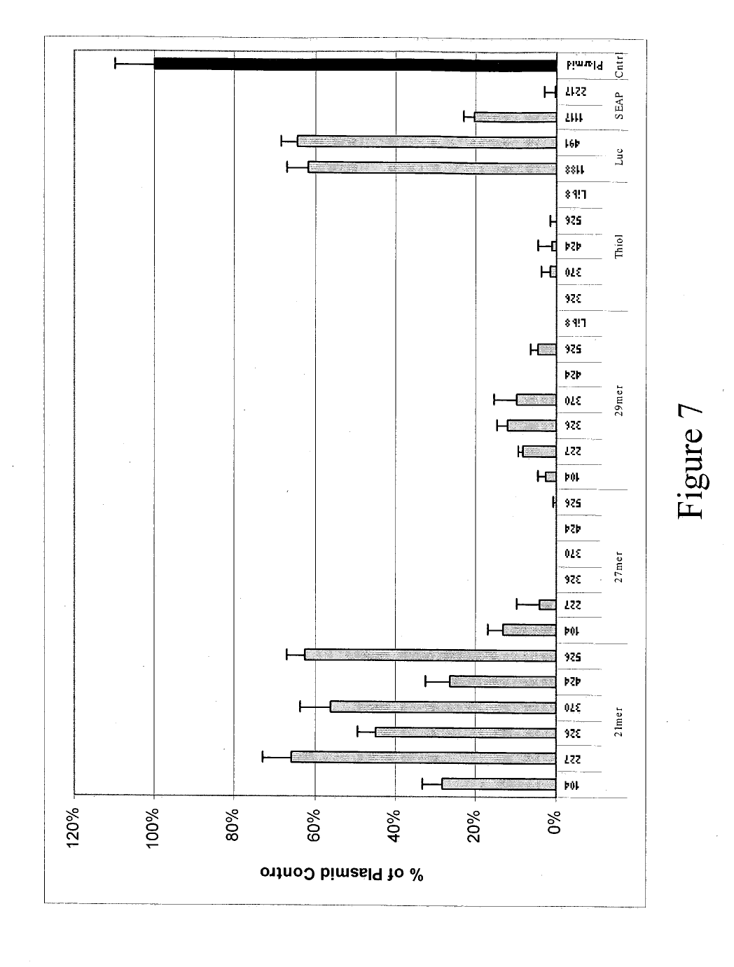

The phenomenon of siRNA-directed promoter silencing is not limited to human kidney HEK 293 cells. Human ovarian cancer cells (HeLa cells) were co-transfected with a vector having the firefly luciferase gene with transcription driven by the CMV promoter and a variety of siRNAs directed against the CMV promoter, including 29mers having duplex regions of 27 nucleotide unit, 27mers having duplex regions of 25 nucleotide units, and 21mers having duplex regions of 19 nucleotide units and phosphorothioate modified internucleotide linkages (see FIG. 6). At 24 hours post-transfection, individual unmodified 21mers exhibited from about 10 to about 55% silencing, whereas individual 27mers having duplex regions of 25 nucleotide units exhibited about 85 to more than 90% silencing, 29mers having duplex regions of 27 nucleotide units exhibited from about 80 to more than 90% silencing, and 21mers having 19mer duplex regions and phosphorothioate modified internucleotide linkages exhibited from about 85% to 100% silencing individually, and 100% silencing when pooled (see FIG. 6). One hundred percent silencing was also observed with pooled 19mers having phosphorothioate modified internucleotide linkages. The experiment was repeated, using a vector wherein transcription of the reporter gene secreted human alkaline phosphatase is driven by the CMV promoter, and similar results were observed (see FIG. 7). It should be noted that silencing was observed in the promoter targeted, and did not affect cyclophyllin, a non-targeted endogenous gene. The sequences of the siRNAs used in the experiment illustrated in FIGS. 6 and 7 are listed in Table 6.

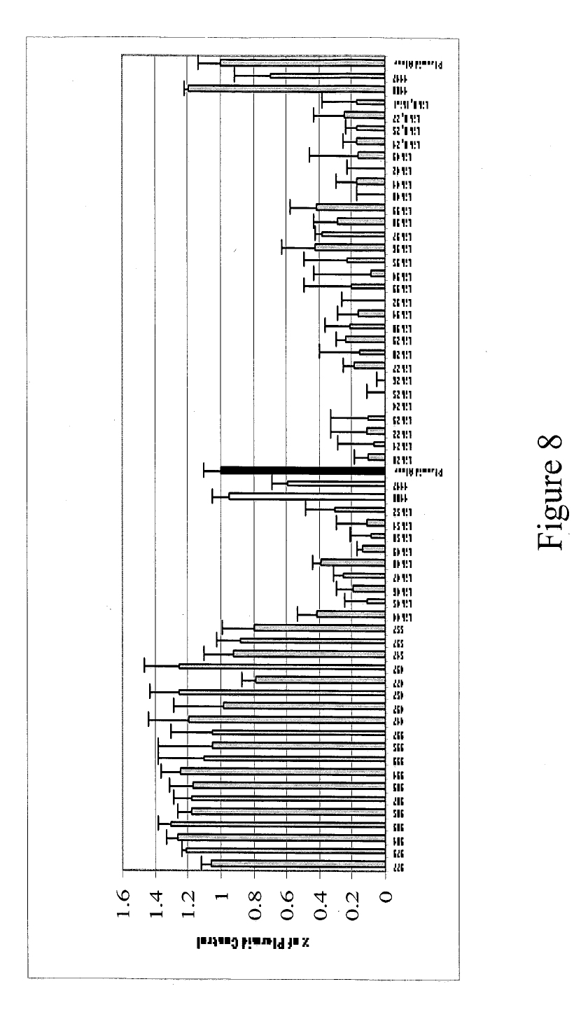

The effect of pooling siRNAs directed against non-protein coding sequences such as promoters is underscored by FIG. 8. FIG. 8 illustrates an experiment wherein individual 21mers having 19mer duplex regions directed against the CMV promoter were co-transfected individually with a CMV-SEAP vector, and in pools, into human kidney HEK 293 cells, and reporter activity was assayed after 24 hours. The content of the siRNA in the pools is indicated in Tables 7A and 7B . Individual 21mers having 19mer duplex regions were relatively ineffective at silencing the CMV promoter, whereas pools of 21mers having 19mer duplex regions silenced the promoter from about 40 to about 100%, depending upon the pool. Thus, simultaneous transfection of multiple siRNAs directed against a non-protein coding sequence such as a promoter are required for effective transcriptional silencing. Preferably, at least two siRNAs directed against a non-protein coding sequence are employed. More preferably, four or more siRNAs are employed. Most preferably, at least eight or more siRNAs are employed.

According to a fourth embodiment, the present invention provides a method of gene silencing comprising introducing at least two siRNA molecules into a mammalian cell, wherein said at least two siRNA molecules are each comprised of a sense strand, an antisense strand that is at least substantially complementary to a region of non-protein coding target nucleic acid sequence, and a nucleus uptake modification located within at least one of said sense strand and said antisense strand. Additionally, the antisense strand of each of said at least two siRNA molecules is at least substantially complementary to a different region of the non-protein coding target nucleic acid sequence. By a different region is meant that the at least two siRNA molecules do not completely overlap, that is, they do not have the same sequence.

Preferred nucleus uptake modifications are the same as those in the third embodiment. Preferably, the antisense strand is 100% complementary to the region of non-protein coding target nucleic acid sequence. The sense strand is preferably substantially complementary to the region of the antisense strand with which it forms a duplex (excluding overhang regions, if present). More preferably, the sense strand is 100% complementary to the region of the antisense strand with which it forms a duplex. The antisense strand and/or sense strand may have overhang regions of any length. If they have overhang regions, these regions are preferably 6 nucleotides or fewer in length, more preferably 3 nucleotides or fewer in length and most preferably two nucleotides in length. The nucleotides of at least one siRNA, or at least one strand of a duplex siRNA, may be modified or unmodified. Preferably, the non-coding target nucleic acid sequence comprises a regulatory sequence. The regulatory sequence preferably comprises one or more sequences selected from the group consisting of promoters and enhancers. The duplex region of the siRNA molecule preferably comprises between 26 and 29 base pairs.

As in the first through third embodiments, the siRNA may also contain stabilization modifications, may be synthesized, and may be introduced into the mammalian cell as described in connection with the above embodiments.

Once synthesized, the polynucleotides of the present invention may immediately be used or be stored for future use. Preferably, the polynucleotides of the invention are stored as duplexes in a suitable buffer. Many buffers are known in the art suitable for storing siRNAs. For example, the buffer may be comprised of 100 mM KCl, 30 mM HEPES-pH 7.5, and 1 mM MgCl₂. Preferably, the double stranded polynucleotides of the present invention retain 30% to 100% of their activity when stored in such a buffer at 4° C. for one year. More preferably, they retain 80% to 100% of their biological activity when stored in such a buffer at 4° C. for one year. Alternatively, the compositions can be stored at −20° C. in such a buffer for at least a year or more. Preferably, storage for a year or more at −20° C. results in less than a 50% decrease in biological activity. More preferably, storage for a year or more at −20° C. results in less than a 20% decrease in biological activity after a year or more. Most preferably, storage for a year or more at −20° C. results in less than a 10% decrease in biological activity.

In order to ensure stability of the siRNA pools prior to usage, they may be retained in dried-down form at −20° C. until they are ready for use. Prior to usage, they should be resuspended; however, even once resuspended, for example, in the aforementioned buffer, they should be kept at −20° C. until used. The aforementioned buffer, prior to use, may be stored at approximately 4° C. or room temperature. Effective temperatures at which to conduct transfection are well known to persons skilled in the art, but include for example, room temperature.

Because the ability of the modified dsRNAs of the present invention to retain functionality and to resist degradation is not dependent on the sequence of the bases, the cell type, or the species into which it is introduced, the present invention is applicable across a broad range of mammals, including but not limited to humans. The present invention is particularly advantageous for use in mammals such as cattle, horse, goats, pigs, sheep, canines, rodents such as hamsters, mice, and rats, and primates such as, for example, gorillas, chimpanzees, and humans.

The present invention may be used advantageously with diverse cell types include those of the germ cell line, as well as somatic cells. The cells may be stem cells or differentiated cells. For example, the cell types may be embryonic cells, oocytes sperm cells, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes and cells of the endocrine or exocrine glands.

The present invention is applicable for use for employing RNA interference against a broad range of genes, including but not limited to the 45,000 genes of a human genome, such as those implicated in diseases such as diabetes, Alzheimer's and cancer, as well as all genes in the genomes of the aforementioned organisms.

The compositions and methods of the present invention may be administered to a cell or applied by any method that is now known or that comes to be known and that from reading this disclosure, one skilled in the art would conclude would be useful with the present invention. For example, the polynucleotides may be passively delivered to cells.

Passive uptake of modified polynucleotides can be modulated, for example, by the presence of a conjugate such as a polyethylene glycol moiety or a cholesterol moiety at the 5′ terminal of the sense strand and/or, in appropriate circumstances, a pharmaceutically acceptable carrier.

Preferably, the polynucleotides are double-stranded when they are administered.

The stabilized dsRNAs of the present invention may be used in a diverse set of applications, including but not limited to basic research, drug discovery and development, diagnostics and therapeutics. For example, the present invention may be used to validate whether a gene product is a target for drug discovery or development. In this application, the RNA that corresponds to a target nucleic acid sequence of interest is identified for silencing. One or more polynucleotides that are specific for targeting the regulatory sequence of the particular target sequence are introduced into a cell or organism, preferably in double-stranded form. The cell or organism is maintained under conditions allowing for the methylation of the targeted RNA and/or methylation of nuclear proteins such as, for example, one or more histones, resulting in decreased activity or transcription of a gene. The extent of any decreased activity, such as, for example, transcription or translation, of the gene is then measured, along with the effect of such decreased activity, and a determination is made that if activity is decreased, then the nucleic acid sequence of interest is a target for drug discovery or development. In this manner, phenotypically desirable effects can be associated with RNA interference of particular target nucleic acids of interest, and in appropriate cases toxicity and pharmacokinetic studies can be undertaken and therapeutic preparations developed.

The present invention may also be used in RNA interference applications that induce transient or permanent states of disease or disorder in an organism by, for example, attenuating the activity of a target nucleic acid of interest believed to be a cause or factor in the disease or disorder of interest. Increased activity of the target nucleic acid of interest may render the disease or disorder worse, or tend to ameliorate or to cure the disease or disorder of interest, as the case may be. Likewise, decreased activity of the target nucleic acid of interest may cause the disease or disorder, render it worse, or tend to ameliorate or cure it, as the case may be. Target nucleic acids of interest can comprise genomic or chromosomal nucleic acids or extrachromosomal nucleic acids, such as viral nucleic acids. Target nucleic acids of interest can include all manner of nucleic acids, such as, for example, non-coding DNA, regulatory DNA, repetitive DNA, reverse repeats, centromeric DNA, DNA in euchromatin regions, DNA in heterochromatin regions, promoter sequences, enhancer sequences, introns sequences, exon sequences, and the like.

Further, the present invention may be used in RNA interference applications that determine the function of a target nucleic acid or target nucleic acid sequence of interest. For example, knockdown experiments that reduce or eliminate the activity of a certain target nucleic acid of interest, such as a promoter or promoter region, enhancer, transcription factor binding site, and the like, can be performed. This can be achieved by performing RNA interference with one or more siRNAs targeting a particular target nucleic acid of interest. Observing the effects of such a knockdown can lead to inferences as to the function of the target nucleic acid of interest. RNA interference can also be used to examine the effects of polymorphisms, such as biallelic polymorphisms, by attenuating the activity of a target nucleic acid of interest having one or the other allele, and observing the effect on the organism or system studied. Therapeutically, one allele or the other, or both, may be selectively silenced using RNA interference where selective allele silencing is desirable.

Still further, the present invention may be used in RNA interference applications, such as diagnostics, prophylactics, and therapeutics. For these applications, an organism suspected of having a disease or disorder that is amenable to modulation by manipulation of a particular target nucleic acid of interest is treated by administering siRNA. Results of the siRNA treatment may be ameliorative, palliative, prophylactic, and/or diagnostic of a particular disease or disorder. Preferably, the siRNA is administered in a pharmaceutically acceptable manner with a pharmaceutically acceptable carrier with or without a diluent.

Therapeutic applications of the present invention can be performed with a variety of therapeutic compositions and methods of administration. Pharmaceutically acceptable carriers and diluents are known to persons skilled in the art. Methods of administration to cells and organisms are also known to persons skilled in the art. Dosing regimens, for example, are known to depend on the severity and degree of responsiveness of the disease or disorder to be treated, with a course of treatment spanning from days to months, or until the desired effect on the disorder or disease state is achieved. Chronic administration of siRNAs may be required in certain cases for lasting desired effects with some diseases or disorders. Suitable dosing regimens can be determined by, for example, administering varying amounts of one or more siRNAs in a pharmaceutically acceptable carrier or diluent, by a pharmaceutically acceptable delivery route, and amount of drug accumulated in the body of the recipient organism can be determined at various times following administration. Similarly, the desired effect (for example, degree of suppression of transcription or expression or activity of a gene product or gene activity) can be measured at various times following administration of the siRNA, and this data can be correlated with other pharmacokinetic data, such as body or organ accumulation. Those of ordinary skill can determine optimum dosages, dosing regimens, and the like. Those of ordinary skill may employ EC₅₀ data from in vivo and in vitro animal models as guides for human studies.

Further, the polynucleotides can be administered in a cream or ointment topically, an oral preparation such as a capsule or tablet or suspension or solution, and the like. The route of administration may be intravenous, intramuscular, dermal, subdermal, cutaneous, subcutaneous, intranasal, oral, rectal, by eye drops, by tissue implantation of a device that releases the siRNA at an advantageous location, such as near an organ or tissue or cell type harboring a target nucleic acid of interest.

Having described the invention with a degree of particularity, examples will now be provided. These examples are not intended to and should not be construed to limit the scope of the claims in any way. Although the invention may be more readily understood through reference to the following examples, they are provided by way of illustration and are not intended to limit the present invention unless specified.

Although the invention has been described and has been illustrated in connection with certain specific or preferred inventive embodiments, it will be understood by those of skill in the art that the invention is capable of many further modifications. This application is intended to cover any and all variations, uses, or adaptations of the invention that follow, in general, the principles of the invention and include departures from the disclosure that come within known or customary practice within the art and as may be applied to the essential features described in this application and in the scope of the appended claims.

### EXAMPLES

### Example 1

### Synthesizing Polynucleotides

RNA oligonucleotides were synthesized in a stepwise fashion using the nucleotide addition reaction cycle illustrated in FIG. 9. The synthesis is preferably carried out as an automated process on an appropriate machine. Several such synthesizing machines are known to those of skill in the art. Each nucleotide is added sequentially (3′- to 5′-direction) to a solid support-bound oligonucleotide. Although polystyrene supports are preferred, any suitable support can be used. The first nucleoside at the 3′-end of the chain is covalently attached to a solid support. The nucleotide precursor, an activated ribonucleotide such as a phosphoramidite or H-phosphonate, and an activator such as a tetrazole, for example, S-ethyl-tetrazole (although any other suitable activator can be used) are added (step i in FIG. 9), coupling the second base onto the 5′-end of the first nucleoside. The support is washed and any unreacted 5′-hydroxyl groups are capped with an acetylating reagent such as but not limited to acetic anhydride or phenoxyacetic anhydride to yield unreactive 5′-acetyl moieties (step ii). The P(III) linkage is then oxidized to the more stable and ultimately desired P(V) linkage (step iii), using a suitable oxidizing agent such as, for example, t-butyl hydroperoxide or iodine and water. At the end of the nucleotide addition cycle, the 5′-silyl group is cleaved with fluoride ion (step iv), for example, using triethylammonium fluoride or t-butyl ammonium fluoride. The cycle is repeated for each subsequent nucleotide. It should be emphasized that although FIG. 9 illustrates a phosphoramidite having a methyl protecting group, any other suitable group may be used to protect or replace the oxygen of the phosphoramidite moiety. For example, alkyl groups, cyanoethyl groups, or thio derivatives can be employed at this position. Further, the incoming activated nucleoside in step (i) can be a different kind of activated nucleoside, for example, an H-phosphonate, methyl phosphonamidite or a thiophosphoramidite. It should be noted that the initial, or 3′, nucleoside attached to the support can have a different 5′ protecting group such as a dimethoxytrityl group, rather than a silyl group. Cleavage of the dimethoxytrityl group requires acid hydrolysis, as employed in standard DNA synthesis chemistry. Thus, an acid such as dichloroacetic acid (DCA) or trichloroacetic acid (TCA) is employed for this step alone. Apart from the DCA cleavage step, the cycle is repeated as many times as necessary to synthesize the polynucleotide desired.

Following synthesis, the protecting groups on the phosphates, which are depicted as methyl groups in FIG. 9, but need not be limited to methyl groups, are cleaved in 30 minutes utilizing 1 M disodium-2-carbamoyl-2-cyanoethylene-1,1-dithiolate trihydrate (dithiolate) in DMF (dimethylformamide). The deprotection solution is washed from the solid support bound oligonucleotide using water. The support is then treated with 40% methylamine for 20 minutes at 55° C. This releases the RNA oligonucleotides into solution, deprotects the exocyclic amines and removes the acetyl protection on the 2′-ACE groups. The oligonucleotides can be analyzed by anion exchange HPLC at this stage.

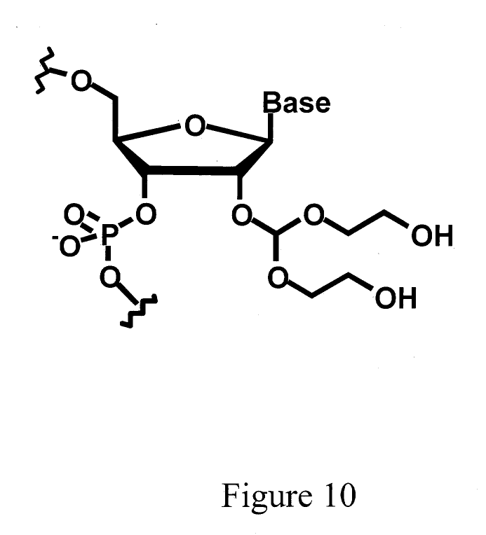

The 2′-orthoester groups are the last protecting groups to be removed, if removal is desired. The structure of the 2′-ACE protected RNA immediately prior to 2′-deprotection is represented in FIG. 10. For the siRNAs used in the experiments described herein, the 2′-orthoester groups were removed.

For automated procedures, solid supports having the initial nucleoside are installed in the synthesizing instrument. The instrument will contain all the necessary ancillary reagents and monomers needed for synthesis. Reagents are maintained under argon, since some monomers, if not maintained under an inert gas, can hydrolyze. The instrunent is primed so as to fill all lines with reagent. A synthesis cycle is designed that defines the delivery of the reagents in the proper order according to the synthesis cycle, delivering the reagents in the order specified in FIG. 9. Once a cycle is defined, the amount of each reagent to be added is defined, the time between steps is defined, and washing steps are defined, synthesis is ready to proceed once the solid support having the initial nucleoside is added.

Modification can be achieved through three different general methods. The first, which is implemented for carbohydrate and base modifications, as well as for introduction of certain linkers and conjugates, employs modified phosphoramidites in which the modification is pre-existing. An example of such a modification would be the carbohydrate 2′-modified species (2′-F, 2′-NH₂, 2′-O-alkyl, etc.) wherein the 2′ orthoester is replaced with the desired modification. 3′ or 5′ terminal modifications could also be introduced such as fluoroscein derivatives, Dabsyl, cholesterol, cyanine derivatives or polyethylene glycol. Certain inter-nucleotide bond modifications can also be introduced via the incoming reactive nucleoside intermediate. Examples of the resultant internucleotide bond modification include but are not limited to methylphosphonates, phosphoramidates, phosphorothioates or phosphorodithioates.

Many modifiers can be employed using the same or similar cycles. Examples in this class would include, for example, 2-aminopurine, 5-methyl cytidine, 5-aminoallyl uridine, diaminopurine, 2-O-alkyl, multi-atom spacers, single monomer spacers, 2′-aminonucleosides, 2′-fluoro nucleosides, 5-iodouridine, 4-thiouridine, acridines, 5-bromouridine, 5-fluorocytidine, 5-fluorouridine, 5-iodouridine, 5-iodocytidine, 5-biotin-thymidine, 5-fluoroscein -thymidine, inosine, pseudouridine, a basic monomer, nebularane, deazanucleoside, pyrene nucleoside, azanucleoside, etc. Often the rest of the steps in the synthesis would remain the same with the exception of modifications that introduce substituents that are labile to standard deprotection conditions. Here modified conditions would be employed that do not effect the substituent. Second, certain internucleotide bond modifications require an alteration of the oxidation step to allow for their introduction. Examples in this class include phosphorothioates and phosphorodithioates wherein oxidation with elemental sulfur or another suitable sulfur transfer agent is required. Third, certain conjugates and modifications are introduced by “post-synthesis” process, wherein the desired molecule is added to the biopolymer after solid phase synthesis is complete. An example of this would be the addition of polyethylene glycol to a pre-synthesized oligonucleotide that contains a primary amine attached to a hydrocarbon linker. Attachment in this case can be achieved by using a N-hydroxy-succinimidyl ester of polyethylene glycol in a solution phase reaction.

While this outlines the most preferred method for synthesis of synthetic RNA and its analogs, any nucleic acid synthesis method which is capable of assembling these molecules could be employed in their assembly. Examples of alternative methods include 5′-DMT-2′-TBDMS and 5′-DMT-2′-TOM synthesis approaches. Some 2′-O-methyl, 2′-F and backbone modifications can be introduced in transcription reactions using modified and wild type T7 and SP6 polymerases, for example.

### Synthesizing Modified RNA

The following guidelines are provided for synthesis of modified RNAs, and can readily be adapted to use on any of the automated synthesizers known in the art.

### 3′ Terminal Modifications

There are several methods for incorporating 3′ modifications. The 3′ modification can be anchored or “loaded” onto a solid support of choice using methods known in the art. Alternatively, the 3′ modification may be available as a phosphoramidite. The phosphoramidite is coupled to a universal support using standard synthesis methods where the universal support provides a hydroxyl at which the 3′ terminal modification is created by introduction of the activated phosphoramidite of the desired terminal modification. Alternatively, the 3′ modification could be introduced post synthetically after the polynucleotide is removed from the solid support. The free polynucleotide initially has a 3′ terminal hydroxyl, amino, thiol, or halogen that reacts with an appropriately activated form of the modification of choice. Examples include but are not limited to N-hydroxy succinimidyl ester, thioether, disulfide, maliemido, or haloalkyl reactions. This modification now becomes the 3′ terminus of the polynucleotide. Examples of modifications that can be conjugated post synthetically can be but are not limited to fluorosceins, acridines, TAMRA, dabsyl, cholesterol, polyethylene glycols, multi-atom spacers, cyanines, lipids, carbohydrates, fatty acids, steroids, peptides, or polypeptides,

### 5′ Terminal Modifications

There are a number of ways to introduce a 5′ modification into a polynucleotide. For example, a nucleoside having the 5′ modification can be purchased and subsequently activated to a phosphoramidite, for example. The phosphoramidite having the 5′ modification may also be commercially available. Then, the activated nucleoside having the 5′ modification is employed in the cycle just as any other activated nucleoside may be used. However, not all 5′ modifications are available as phosphoramidites. In such an event, the 5′ modification can be introduced in an analogous way to that described for 3′ modifications above.

### Thioates

Polynucleotides having one or more thioate moieties, such as phosphorothioate linkages, were made in accordance with the synthesis cycle described above and illustrated in FIG. 9. However, in place of the t-butyl hydroperoxide oxidation step, elemental sulfur or another sulfurizing agent was used.

### 5′-Thio Modifications

Monomers having 5′ thiols can be purchased as phosphoramidites from commercial suppliers such as Glen Research. These 5′ thiol modified monomers generally bear trityl protecting groups. Following synthesis, the trityl group can be removed by any method known in the art.

### Other Modifications

For certain modifications, the steps of the synthesis cycle will vary somewhat. For example, where the 3′ end has an inverse dT (wherein the first base is attached to the solid support through the 5′-hydroxyl and the first coupling is a 3′-3′ linkage) detritylation and coupling occurs more slowly, so extra detritylating reagent, such as dichloroactetic acid (DCA), should be used and coupling time should be increased to 300 seconds. Some 5′ modifications may require extended coupling time. Examples include cholesterol, fluorophores such as Cy3 or Cy5 biotin, dabsyl, amino linkers, thio linkers, spacers, polyethylene glycol, phosphorylating reagent, BODIPY, or photocleavable linkers.

It should be noted that if a polynucleotide is to have only a single modification, that modification can be most efficiently carried out manually by removing the support having the partially built polynucleotide on it, manually coupling the monomer having the modification, and then replacing the support in the automated synthesizer and resuming automated synthesis.

### Example 2

### Deprotection and Cleavage of Synthesized Oligos from the Support

Cleaving can be done manually or in an automated process on a machine. Cleaving of the protecting moiety from the internucleotide linkage, for example a methyl group, can be achieved by using any suitable cleaving agent known in the art, for example, dithiolate or thiophenol. One molar dithiolate in DMF is added to the solid support at room temperature for 10 to 20 minutes. The support is then thoroughly washed with, for example, DMF, then water, then acetonitrile. Alternatively a water wash followed by a thorough acetonitrile will suffice to remove any residual dithioate.

Cleavage of the polynucleotide from the support and removal of exocyclic base protection can be done with 40% aqueous N-methylamine (NMA), followed by heating to 55 degrees Centigrade for twenty minutes. Once the polynucleotide is in solution, the NMA is carefully removed from the solid support. The solution containing the polynucleotide is then dried down to remove the NMA under vacuum. Further processing, including duplexing, desalting, gel purifying, quality control, and the like can be carried out by any method known in the art.

For some modifications, the NMA step may vary. For example, for a 3′ amino modification, the treatment with NMA should be for forty minutes at 55 degrees Centigrade. Puromycin, 5′ terminal amino linker modifications, and 2′ amino nucleoside modifications are heated for 1 hour after addition of 40% NMA. Oligonucleotides modified with Cy5 are treated with ammonium hydroxide for 24 hours while protected from light.

### Preparation of Cleave Reagents

HPLC grade water and synthesis grade acetonitrile are used. Four and a half grams of dithiolate crystals are added to 90 mL of DMF. Forty percent NMA can be purchased, ready to use, from a supplier such as Sigma Aldrich Corporation.

### Annealing Single Stranded Polynucleotides to Produce Double Stranded siRNA

Single stranded polynucleotides can be annealed by any method known in the art, employing any suitable buffer. For example, equal amounts of each strand can be mixed in a suitable buffer, such as, for example, 50 mM HEPES pH 7.5, 100 mM potassium chloride, 1 mM magnesium chloride. The mixture is heated for one minute at 90 degrees Centigrade, and allowed to cool to room temperature. In another example, each polynucleotide is separately prepared such that each is at 50 micromolar concentration. Thirty microliters of each polynucleotide solution is then added to a tube with 15 microliters of 5× annealing buffer, wherein the annealing buffer final concentration is 100 mM potassium chloride, 30 mM HEPES-KOH pH 7.4 and 2 mM magnesium chloride. Final volume is 75 microliters. The solution is then incubated for one minute at 90 degrees Centigrade, spun in a centrifuge for 15 seconds, and allowed to incubate at 37 degrees Centigrade for one hour, then allowed to come to room temperature. This solution can then be stored frozen at minus 20 degrees Centigrade and freeze thawed up to five times. The final concentration of the duplex is 20 micromolar. An example of a buffer suitable for storage of the polynucleotides is 20 mM KCl, 6 mM HEPES pH 7.5, 0.2 mM MgCl₂. All buffers used should be RNase free.

### Removal of the Orthoester Moiety

The orthoester moiety or moieties may be removed from the polynucleotide by any suitable method known in the art. One such method employs a volatile acetic acid-tetramethylenediamine (TEMED) pH 3.8 buffer system that can be removed by lyophilization following removal of the orthoester moiety or moieties. Deprotection at a pH higher than 3.0 helps minimize the potential for acid-catalyzed cleavage of the phosphodiester backbone. For example, deprotection can be achieved using 100 mM acetic acid adjusted to pH 3.8 with TEMED by suspending the orthoester protected polynucleotide and incubating it for 30 minutes at 60 degrees Centigrade. The solution is then lyophilized or subjected to a SpeedVac to dryness prior to use. If necessary, desalting following deprotection can be performed by any method known in the art, for example, ethanol precipitation or desalting on a reversed phase cartridge.

### Example 3

### Performing RNA Interference

### Transfection

SiRNA duplexes were annealed using standard buffer (50 millimolar HEPES pH 7.5, 100 millimolar KCl, 1 mM MgCl₂). The transfections are done according to the standard protocol described below.

### Standard Transfection Protocol for 96 Well and 6 Well Plates: siRNAs

1. Protocols for HEK 293 and HeLa cells are identical.

2. Cell are plated to be 95% confluent on the day of transfection, unless otherwise indicated.

3. SuperRNAsin (Ambion) is added to transfection mixture for protection against RNAses.

4. All solutions and handling have to be carried out in RNAse free conditions.

Plate 1 0.5-1 ml in 25 ml of media in a small flask or 1 ml in 50 ml in a big flask.

### 96 Well Plate

1. Add 3 ml of 0.05% trypsin-EDTA in a medium flask (6 in a big) incubate 5 min at 37 degrees C.

2. Add 7 ml (14 ml big) of regular media and pipet 10 times back and forth to re-suspend cells.

3. Take 25 microliters of the cell suspension from step 2 and 75 microliters of trypan blue stain (1:4) and place 10 microliters in a cell counter.

4. Count number of cells in a standard hemocytometer.

5. Average number of cells×4×10000 is number of cells per ml.

6. Dilute with regular media to have 350 000 /ml.

7. Plate 100 microliters (35000 cell for HEK 293) in a 96 well plate.

### Transfection. For 2×96 well plates (60 well format)

1. OPTI-MEM 2 ml+80 microliters Lipofectamine 2000 (1:25)+15 microliters of SuperRNAsin (AMBION).

2. Transfer iRNA aliquots (0.8 microliters of 100 micromolar to screen (total dilution factor is 1:750, 0.8 microliters of 100 micromolar solution will give 100 nanomolar final) to the dipdish in a desired order (Usually 3 columns×6 for 60 well format or four columns by 8 for 96 well).

3. Transfer 100 microliters of OPTI-MEM.

4. Transfer 100 microliters of OPTI-MEM with Lipofectamine 2000 and SuperRNAsin to each well.

5. Leave for 20-30 min RT.

6. Add 0.55 ml of regular media to each well. Cover plate with film and mix.

7. Array out 100×3×2 directly to the cells (sufficient for two plates).

### Transfection. For 2×6 Well Plates

8. 8 ml OPTI-MEM+160 microliters Lipofectamine 2000 (1:25). 30 microliters of SuperRNAsin (AMBION).

9. Transfer iRNA aliquots (total dilution factor is 1:750, 5 microliters of 100 micromolar solution will give 100 nanomolar final) to polystyrene tubes.

10. Transfer 1300 microliters of OPTI-MEM with Lipofectamine 2000 and SuperRNAsin (AMBION).

11. Leave for 20-30 min RT.

12. Add 0.55 ml of regular media to each well. Cover plate with film and mix.

13. Transfer 2 ml to each well (sufficient for two wells).

The mRNA or protein levels are measured 24, 48, 72, and 96 hours post transfection with standard kits or Custom B-DNA sets and QuantiGene kits (Bayer).

### Example 4

### Measurement of Activity/Detection

The level of siRNA-induced RNA interference, or gene silencing, was estimated by assaying the reduction in target mRNA levels or reduction in the corresponding protein levels. Assays of mRNA levels were carried out using B-DNA™ technology (QuantiGene Corp.). Protein levels for firefly luciferase were assayed by STEADY GLO™ kits (Promega Corp.). Human alkaline phosphatase levels were assayed by Great EscAPe SEAP Fluorescence Detection Kits (#K2043-1), BD Biosciences, Clontech.

### Example 5

### Performing RNA Interference using Individual 21mers Directed Against the CMV Promoter

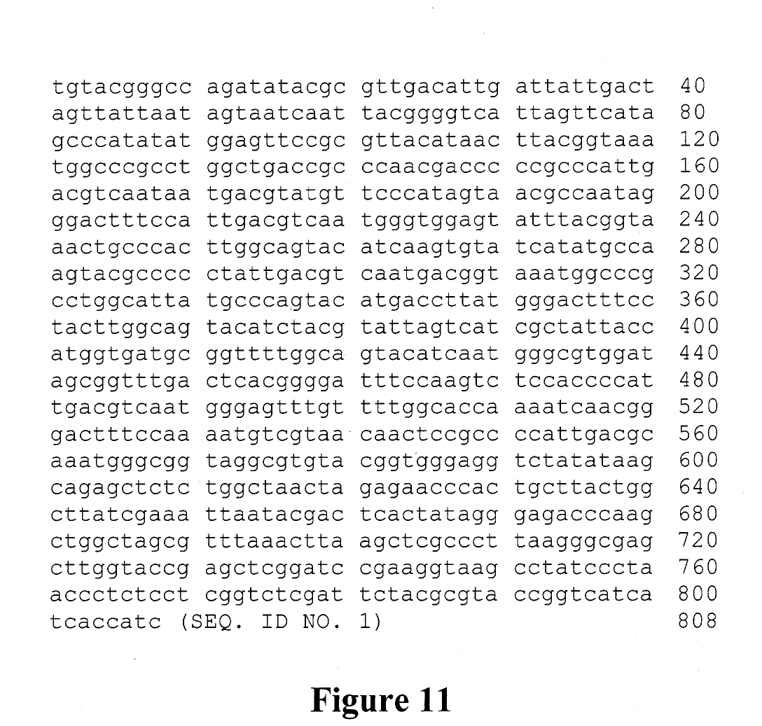

Each of the siRNAs used in the studies are represented herein as sense strands having a di-dT at the 3′ end. It should be understood that duplex siRNAs were employed in the studies described herein, wherein the antisense strand also has a di-dT at the 3′ end. In each case, it is the sense strand that is homologous to the CMV promoter (for the CMV promoter sequence, see FIG. 11). The term “21mer,” refers to the number of nucleotides that make up each strand of an siRNA; herein, a double-stranded 21mer comprises a 19mer duplex region and a di-dT overhang at each end of the duplex. The term “27mer,” refers to a double stranded siRNA having a duplex region of 25 nucleotide units and a di-dT overhang at the 3′ end of the sense strand and at the 3′ end of the antisense strand. The term “29mer” refers to a double stranded siRNA having a duplex region of 27 nucleotide units and a di-dT overhang at the 3′ end of the sense strand and at the 3′ end of the antisense strand.

The indication “Start” in the Tables herein is used as a reference point with relation to the CMV promoter region. By way of example, a “Start” indication of a 21mer designated 104 (see, for example, Table 1) indicates that the sense strand of the 19mer duplex region of the siRNA is homologous to the CMV promoter region in the following manner: the 5′ end of the sense strand reflects the 104^{th} nucleotide of the CMV promoter, when read 5′ to 3′, and extends 19 nucleotide units in the 3′ direction. Thus, the siRNA is homologous to positions 104 through 122, with the exception that U's are substituted for T's. For siRNA duplexes longer than 21mers, the “Start” designation indicates that the siRNA duplex is homologous to 19 nucleotides in the 3′ direction along the CMV promoter region beginning at the “Start” point, and a fixed number of nucleotides upstream, in the 5′ direction, from the “Start” designation. By way of example, a 27mer having a 25mer duplex region, designated 326 (see, for example, Table 4), means that the 27mer is homologous to 19 nucleotides in the 3′ direction along the CMV promoter region from positions 326 through 144, and since it is a 27mer it has six additional nucleotides (for a total of 25) in its duplex region, it is also homologous to CMV promoter positions 320 through 325, six nucleotide units in the 5′ direction from the “Start” designation. Therefore, a 27mer designated 326 has a duplex region homologous to positions 320 through 344 of the CMV promoter region. Similarly, a 29mer has a duplex region of 27 nucleotides. Thus, a 29mer designated 326 (see, for example, Table 4) is homologous to positions 318 through 344 of the CMV promoter region, read 5′ to 3′. In this way, as the length of the duplex region of the double-stranded siRNA increases, the additional length is achieved by the addition of nucleotide units to the 3′ region of the antisense strand that are complementary to the CMV promoter, but before the antisense strand's di-dT terminus.

As an initial non-protein coding nucleic acid sequence target, the human cytomegalovirus (CMV) promoter region was chosen. Because the promoter region can be engineered to drive the expression of several reporter genes in an appropriate vector, it is an ideal target for ease of analysis. The sequence of the CMV promoter region is provided in FIG. 11, oriented 5′ to 3′. The numerals to the right denote nucleotide number, numbered from the first nucleotide of the CMV promoter region and counting from the 5′ direction to the 3′ direction.

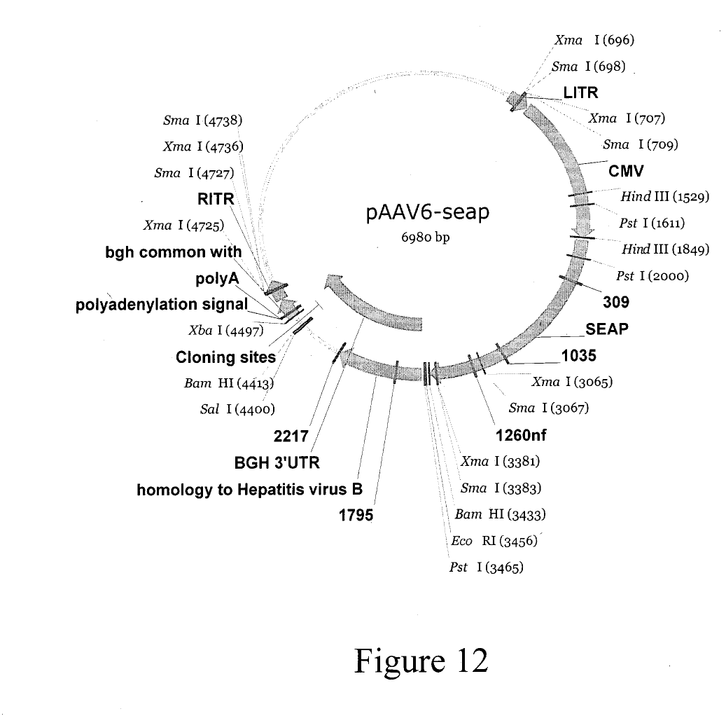

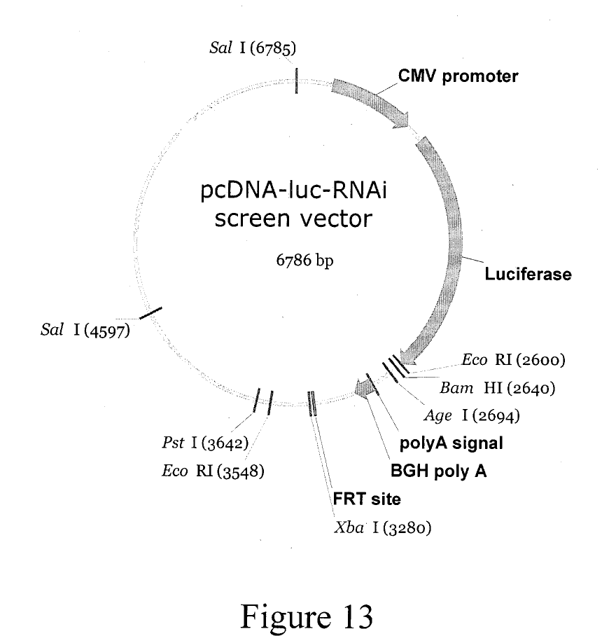

The CMV promoter, driving transcription of human secreted alkaline phosphatase (SEAP) was used as a target in the experiments described herein. This construct is denoted herein as CMV-SEAP vector, or pAAV6, shown in FIG. 12. Also used, where indicated, was the CMV promoter driving transcription of the firefly luciferase gene (FfLuc), denoted herein as CMV-fLuc vector. This vector is shown in FIG. 13. siRNA duplexes were synthesized that were directed against sequences of the CMV promoter region, but not against the SEAP reporter gene. Initially, fourteen naked, or unmodified, duplexes were initially screened in human kidney HEK 293 cells independently and in pools. For the initial study, the duplexes were not selected so as to have CpG sequences (typical methylation targets). Furthermore, data obtained suggests that CpG sequences in the siRNA duplexes, or the corresponding target nucleic acids, may not be necessary for silencing. Pools resulting in the most significant silencing do not correlate significantly with CpG content. The fourteen duplexes initially screened are listed in Table 1.

TABLE 1

Initial 21mers Having 19mer
Duplex Regions Screened in Human
Kidney HEK 293 Cells

| | | |
|---|---|---|
| Start | Sequence | SEQ. ID. NO. |

| | | |
|---|---|---|
| 1 | uguacgggccagauauacgdTdT | 2 |
| 12 | gauauacgcguugacauugdTdT | 3 |
| 32 | uuauugacuaguuauuaaudTdT | 4 |
| 57 | caauuacggggucauuagudTdT | 5 |
| 104 | acauaacuuacgguaaaugdTdT | 6 |
| 175 | guauguucccauaguaacgdTdT | 7 |
| 227 | gacuauuuacgguaaacugdTdT | 8 |
| 268 | guaucauaugccaaguacgdTdT | 9 |
| 326 | cauuaugcccaguacaugadTdT | 10 |
| 370 | guacaucuacguauuagucdTdT | 11 |
| 424 | caucaaugggcguggauagdTdT | 12 |
| 526 | uccaaaaugucguaacaacdTdT | 13 |
| 576 | guguacggugggaggucuadTdT | 14 |
| 618 | cuagagaacccacugcuuadTdT | 15 |

The siRNAs in Table 1 were transfected as duplexes having di-dT overhangs on both 5′ and 3′ ends of the duplexes, with complementary strands (bearing 3′ di-dTs), into human kidney HEK 293 cells in 96 well plates as described herein. The activity of the reporter gene SEAP was measured at 24, 48 and 72 hours following transfection. Controls were run that included transfection of vector alone (i.e., without siRNA), and transfection of an inverted luciferase construct in the CMV-containing vector as a nonsense control. Results are shown in FIGS. 1A, 1B and 1C.

### Example 6

### Performing RNA Interference using Pools of 21mers Directed Against Promoter Regions

RNA interference was performed using pools of the 21mers having 19mer duplex regions listed in Table 1, wherein the duplexes were pooled into libraries of 21mers. The siRNAs were transfected at two concentrations: one micromolar, and one-tenth micromolar. The composition of each library is listed in Table 1, indicating the sequences by their identifier number. Reference is made to Table 1 for the sequences associated with the 21mers of the pools. For example, Library 1 contained all the 21mers listed in Table 1; Library 2 contained only the 21mers of Table 1 bearing the identifiers 1, 12, 32, 57, 104, 175, and 227; Library 3 contained only the 21mers of Table 1 bearing the identifiers 268, 326, 370, 424, 526, 576 and 618; and so on. Table 1 lists the sequences of the contents of the libraries of Table 2.

TABLE 2
Pools of 21mers with 19mer Duplex Regions and Control

Start

| | |
|---|---|
| Library 1 | 1, 12, 32, 57, 104, 175, 227, 268, 326, 370, 424, 526, 576, 618 |
| Library 2 | 1, 12, 32, 57, 104, 175, 227 |
| Library 3 | 268, 326, 370, 424, 526, 576, 618 |
| Library 4 | 1, 32, 104, 227, 326, 424, 576 |
| Library 5 | 12, 57, 175, 268, 370, 526, 618 |
| Library 6 | 1, 12, 32, 57 |
| Library 7 | 104, 175, 227, 268 |
| Library 8 | 326, 370, 424, 526 |
| Library 9 | 1, 12, 576, 618 |
| Library 10 | 424, 576 |
| Library 11 | 326, 424, 576 |
| Library 12 | 370, 424, 576 |
| Library 13 | 424, 526, 576 |
| S-AS | gugauguaugucagagagudTdT (SEQ. ID NO. 16) |

The siRNA pools in Table 2 were transfected as duplexes having di-dT overhangs on both 5′ and 3′ ends of the duplexes, with complementary strands bearing 3′ di-dTs, into human kidney HEK 293 cells in 96 well plates as described herein. The activity of the reporter gene SEAP was measured at 24, 48 and 72 hours following transfection of the pools. A control was run that included transfection of vector alone (i.e., without siRNA). Results are shown in FIGS. 2A, 2B and 2C.

### Example 7

### Performing RNA Interference using 21mers having Modified Internucleotide Linkages Directed Against Promoter Regions

Four 21mers having 19mer duplex regions and having phosphorothioate internucleotide linkages were synthesized. These 21mers had sequences corresponding to those listed in Table 1 as 326, 370, 424 and 526, but had phosphorothioate modifications at each internucleotide linkage except for the linkage to and between the terminal di-dTs. These four modified 21mers having 19mer duplex regions also had di-dT overhangs at the 5′ and 3′ ends. These 21mers are listed in Table 3.

TABLE 3

21mers with 19mer Duplex Regions
Having Phosphorothioate Modifications

| | | |
|---|---|---|
| Start | Sequence | SEQ. ID. NO. |

| | | |
|---|---|---|
| 326 | c*a*u*u*a*u*g*c*c*c*a*g*u*a*c*a*u*g*adTdT | 17 |
| 370 | g*u*a*c*a*u*c*u*a*c*g*u*a*u*u*a*g*u*cdTdT | 18 |
| 424 | c*a*u*c*a*a*u*g*g*g*c*g*u*g*g*a*u*a*gdTdT | 19 |
| 526 | u*c*c*a*a*a*a*u*g*u*c*g*u*a*a*c*a*a*cdTdT | 20 |

*Indicates a phosphorothioate internucleotide linkage.

The 21mers having 19mer duplex regions and 5′ and 3′ di-dT overhangs, and having phosphorothioate modified internucleotide linkages, were co-transfected with the CMV-SEAP vector into human kidney HEK 293 cells about 12 hours after plating the cells in 96 well plates individually, and in a pool. Two experiments were conducted. In one experiment, the number of cells plated per well was about 10,000. In another experiment, the number of cells plated per well was about 25,000. Activity of the SEAP reporter gene was measured at 24 hours post-transfection for each of the two experiments. In each experiment, the CMV-SEAP vector was transfected in the absence of any siRNA as a positive control.

The results of these experiments using the siRNAs of Table 3 are illustrated in FIG. 3 for cells plated at about 10,000 cells/well (10K) and about 25,000 cells/well (25K).

### Example 8

### Performing RNA Interference using 21, 27 and 29mers Directed Against Promoter Regions

RNA interference was performed using 21mers having 19mer duplex regions, 27mers having 25mer duplex regions and 29mers having 27mer duplex regions using the siRNAs listed in Table 4.

TABLE 4

Duplexes Used to Assess Length
Dependence of Promoter Silencing

| | |
|---|---|
| Duplex | SEQ. |

| | | | |
|---|---|---|---|
| Length | Start | Sequence | ID NO. |

| | | | |
|---|---|---|---|
| 19 | 326 | cauuaugcccaguacaugadTdT | 10 |
| | 370 | guacaucuacguauuagucdTdT | 11 |
| | 424 | caucaaugggcguggauagdTdT | 12 |
| | 526 | uccaaaaugucguaacaacdTdT | 13 |
| 27 | 326 | gccuggcauuaugcccaguacaugadTdT | 21 |
| | 370 | uuggcaguacaucuacguauuagucdTdT | 22 |
| | 424 | gcaguacaucaaugggcguggauagdTdT | 23 |
| | 526 | ggacuuuccaaaaugucguaacaacdTdT | 24 |
| 29 | 326 | ccgccuggcauuaugcccaguacaugadTdT | 25 |
| | 370 | acuuggcaguacaucuacguauuagucdTdT | 26 |
| | 424 | uggcaguacaucaaugggcguggauagdTdT | 27 |
| | 526 | cgggacuuuccaaaaugucguaacaacdTdT | 28 |
| Thio | 326 | c*a*u*u*a*u*g*c*c*c*a*g*u*a*c*a*u*g*adTdT | 17 |
| 19 | 370 | g*u*a*c*a*u*c*u*a*c*g*u*a*u*ux*a*g*u*cdTdT | 18 |
| | 424 | c*a*u*c*a*a*u*g*g*g*c*g*u*g*g*a*u*a*gdTdT | 19 |
| | 526 | u*c*c*a*a*a*a*u*g*u*c*g*u*a*a*c*a*a*cdTdT | 20 |
| Controls | 1117 | tgttcgacgacgccattgadTdT | 29 |
| | 2217 | gugauguaugucagagagudTdT | 16 |

*Indicates a phosphorothioate internucleotide linkage.

In addition to the individual siRNAs described in Table 4, the cells were also transfected with groups of pooled siRNAs having the same length. Thus, all 21mers in Table 4 were transfected as a single pool of duplexes having 19mer duplex regions; all 27mers were transfected as a second pool of duplexes having 25mer duplex regions; all 29mers were transfected as a third pool of duplexes having 27mer duplex regions; and all 21mers having thiophosphate modified internucleotide linkages were transfected as a fourth pool of duplexes having 19mer duplex regions. The results of these experiments are shown in FIG. 4. In FIG. 4, the pools are denoted as “Lib 8” for each of the 21mer group, the 27mer group, the 29mer group, and the 21mer group having phosphorothioate modified internucleotide linkages. The siRNAs directed against the CMV promoter region were co-transfected into human kidney HEK 293 cells, and SEAP reporter gene activity was measured after 24 hours. A positive control containing CMV and SEAP, without siRNA treatment, was run. Two other controls were run. Control 1117 was run with an inefficient siRNA against the coding region of the SEAP reporter gene. Control 2217 was run with an efficient siRNA targeted against the coding region of the SEAP reporter gene.

### Example 9

### Performing RNA Interference using 27mers Directed Against Promoter Regions: Effect of Cell Growth

Four 27mers having 25mer duplex regions were individually co-transfected with the CMV-SEAP vector under two different cell growth conditions of human kidney HEK 293 cells. A pool of four siRNAs directed against regions in the CMV promoter were also co-transfected with the CMV-SEAP vector. In the first condition, cells were plated at a density of about 10,000 cells per well in 96 well plates. In the second condition, cells were plated at a density of about 25,000 cells per well. Transfections were made at about 12 hours after plating, and reporter activity was measured 24 hours after transfection. The 27mers having 25mer duplex regions transfected in this experiment are listed in Table 5.

TABLE 5

27mer siRNAs Employed to Investigate
Growth Dependence of Silencing

SEQ.

| | | |
|---|---|---|
| Start | Sequence | ID. NO. |

| | | |
|---|---|---|
| 326 | gccuggcauuaugcccaguacaugadTdT | 21 |
| 370 | uuggcaguacaucuacguauuagucdTdT | 22 |
| 424 | gcaguacaucaaugggcguggauagdTdT | 23 |
| 526 | ggacuuuccaaaaugucguaacaacdTdT | 24 |
| Library 8 | 326, 370, 424 and 526 | |

As a positive control, the CMV-SEAP vector was transfected alone. The results of this experiment are illustrated in FIG. 5.

### Example 10

### Performing RNA Interference in HeLa Cells

Two experiments were conducted using human ovarian cancer cells (HeLa cells). 21mers having 19mer duplex regions, 27mers having 25mer duplex regions, and 29mers having 27mer duplex regions directed against regions of the CMV promoter were individually co-transfected with a CMV-Firefly Luciferase vector (FfLuc CMV). A pool of 29mer siRNAs having 27mer duplex regions were also co-transfected with FfLuc CMV. Four 21mer siRNAs having 19mer duplex regions directed against the CMV promoter, and having phosphorothioate modified internucleotide linkages, were also individually co-transfected with FfLuc CMV. The four 21mers were also co-transfected with FfLuc CMV as a pool. Five controls were also run. Two individual 21mer siRNAs having 19mer duplex regions directed against the firefly luciferase reporter gene were co-transfected with the FfLuc CMV vector (denoted 1188 and 491) as negative controls. Two individual 21mer siRNAs having 19mer duplex regions directed against the secreted human alkaline phosphatase gene (denoted 1117 and 2217) were co-transfected with the FfLuc CMV vector as negative controls. The FfLuc CMV vector was transfected alone as a positive control. The results of this experiment are illustrated in FIG. 6. The experiment was repeated, using the same siRNAs directed against the CMV promoter, but using a CMV-SEAP vector wherein the reporter gene was secreted human alkaline phosphatase. The results of this experiment are illustrated in FIG. 7. The siRNAs used in these experiments are listed in Table 6.

TABLE 6
Duplexes Used to Conduct Silencing in HeLa Cells

| | |
|---|---|
| Duplex | SEQ. ID |

| | | | |
|---|---|---|---|
| Length | Start | Sequence | NO. |

| | | | |
|---|---|---|---|
| 19 | 104 | acauaacuuacgguaaaugdTdT | 6 |
| | 227 | gacuauuuacgguaaacugdTdT | 8 |
| | 326 | cauuaugcccaguacaugadTdT | 10 |
| | 370 | guacaucuacguauuagucdTdT | 11 |
| | 424 | caucaaugggcguggauagdTdT | 12 |
| | 526 | uccaaaaugucguaacaacdTdT | 13 |
| 27 | 104 | cgcguuacauaacuuacgguaaaugdTdT | 30 |
| | 227 | uggguggacuauuuacgguaaacugdTdT | 31 |
| | 326 | gccuggcauuaugcccaguacaugadTdT | 21 |
| | 370 | uuggcaguacaucuacguauuagucdTdT | 22 |
| | 424 | gcaguacaucaaugggcguggauagdTdT | 23 |
| | 526 | ggacuuuccaaaaugucguaacaacdTdT | 24 |
| 29 | 104 | uccgcguuacauaacuuacgguaaaugdTdT | 32 |
| | 227 | aauggguggacuauuuacgguaaacugdTdT | 33 |
| | 326 | ccgccuggcauuaugcccaguacaugadTdT | 34 |
| | 370 | acuuggcaguacaucuacguauuagucdTdT | 35 |
| | 424 | uggcaguacaucaaugggcguggauagdTdT | 36 |
| | 526 | cgggacuuuccaaaaugucguaacaacdTdT | 37 |
| Lib 8 | 326 | c*c*g*c*c*u*g*g*c*a*u*u*a*u*g*c*c*c*a*g*u | 38 |
| 29mer | | *a*c*a*u*g*adTdT | |
| thio | 370 | a*c*u*u*g*g*c*a*g*u*a*c*a*u*c*u*a*c*g*u*a | 39 |
| | | *u*u*a*g*u*cdTdT | |
| | 424 | u*g*g*c*a*g*u*a*c*a*u*c*a*a*u*g*g*g*c*g*u | 40 |
| | | *g*g*a*u*a*gdTdT | |
| | 526 | c*g*g*g*a*c*u*u*u*c*c*a*a*a*a*u*g*u*c*g*u | 41 |
| | | *a*a*c*a*a*cdTdT | |
| | 1188 | gauuauguccgguuauguadTdT | 42 |
| FfLuc | 491 | cugugaauacaaaucacagdTdT | 43 |
| SEAP | 2217 | gugauguaugucagagagudTdT | 16 |

*Indicates a phosphorothioate internucleotide linkage.

### Example 11

### Performing RNA Interference in HeLa Cells

Nineteen siRNAs targeting regions of the CMV promoter were individually co-transfected with the CMV-SEAP construct (vector pAAV6) into HEK 293 cells. In parallel, 33 pools, or libraries, of 4 to 8 siRNAs targeting the CMV promoter, were also co-transfected with the CMV-SEAP construct. The level of reporter (SEAP) expression was measured 24 hours post transfection. Controls run in parallel included the following: an siRNA directed against the coding region of firefly luciferase, as negative control; an siRNA directed against the coding region of the reporter gene human secreted alkaline phosphatase, as an mRNA degradation control; and the CMV-SEAP construct in the absence of any siRNA as a positive control. The results of this experiment are illustrated in FIG. 8. The siRNAs used in the experiment are described in Table 7. In addition to the listing in Table 7, Library 8 was co-transfected with Library 21, Library 25, and Library 27. Library 8, having phosphorothioate modified internucleotide linkages was also used.

TABLE 7A
Pools of Duplex 19mers for Silencing in HeLa Cells

| | Start |
|---|---|
| Library 44 | 377, 385, 395 |
| Library 45 | 379, 387, 397 |
| Library 46 | 377, 397, 417, 437, 457, 477, 497, 517, 537, 557 |
| Library 47 | 377, 397, 417, 437, 457 |
| Library 48 | 397, 417, 437, 457, 477 |
| Library 49 | 417, 437, 457, 477, 497 |
| Library 50 | 437, 457, 477, 497, 517 |
| Library 51 | 457, 477, 497, 517, 537 |
| Library 52 | 477, 497, 517, 537, 557 |
| Library 20 | 377, 379, 381, 383, 385 |
| Library 21 | 379, 381, 383, 385, 387 |
| Library 22 | 381, 383, 385, 387, 389 |
| Library 23 | 383, 385, 387, 389, 391 |
| Library 24 | 385, 387, 389, 391, 393 |
| Library 25 | 387, 389, 391, 393, 395 |
| Library 26 | 389, 391, 393, 395, 397 |
| Library 27 | 377, 379, 381, 383 |
| Library 28 | 379, 381, 383, 385 |
| Library 29 | 381, 383, 385, 387 |
| Library 30 | 383, 385, 387, 389 |
| Library 31 | 385, 387, 389, 391 |
| Library 32 | 387, 389, 391, 393 |
| Library 33 | 389, 391, 393, 395 |
| Library 34 | 391, 393, 395, 397 |
| Library 35 | 377, 379, 381 |
| Library 36 | 379, 381, 383 |
| Library 37 | 381, 383, 385 |
| Library 38 | 383, 385, 387 |
| Library 39 | 385, 387, 389 |
| Library 40 | 387, 389, 391 |
| Library 41 | 389, 391, 393 |
| Library 42 | 391, 393, 395 |
| Library 43 | 393, 395, 397 |

TABLE 7B
Duplexes Used to Conduct Silencing in HeLa Cells

| Start | Sequence | SEQ. ID NO. |
|---|---|---|
| 377 | uacguauuagucaucgcuadTdT | 44 |
| 379 | cguauuagucaucgcuauudTdT | 45 |
| 381 | uauuagucaucgcuauuacdTdT | 46 |
| 383 | uuagucaucgcuauuaccadTdT | 47 |
| 385 | agucaucgcuauuaccaugdTdT | 48 |
| 387 | ucaucgcuauuaccauggudTdT | 49 |
| 389 | aucgcuauuaccauggugadTdT | 50 |
| 391 | cgcuauuaccauggugaugdTdT | 51 |
| 393 | cuauuaccauggugaugcgdTdT | 52 |
| 395 | auuaccauggugaugcggudTdT | 53 |
| 397 | uaccauggugaugcgguuudTdT | 54 |
| 417 | ggcaguacaucaaugggcgdTdT | 55 |
| 437 | ggauagcgguuugacucacdTdT | 56 |
| 457 | gggauuuccaagucuccacdTdT | 57 |
| 477 | ccauugacgucaaugggagdTdT | 58 |
| 497 | uuguuuuggcaccaaaaucdTdT | 59 |
| 517 | acgggacuuuccaaaaugudTdT | 60 |
| 537 | guaacaacuccgccccauudTdT | 61 |
| 557 | acgcaaaugggcgguaggcdTdT | 62 |
| 1188 | gauuauguccgguuauguadTdT | 42 |
| 1117 | tgttcgacgacgccattgadTdT | 29 |

Although the invention has been described and has been illustrated in connection with certain specific or preferred inventive embodiments, it will be understood by those of skill in the art that the invention is capable of many further modifications. This application is intended to cover any and all variations, uses, or adaptations of the invention that follow, in general, the principles of the invention and include departures from the disclosure that come within known or customary practice within the art and as may be applied to the essential features described in this application and in the scope of the appended claims.

What is claimed is:

1. A method of gene silencing comprising introducing at least one siRNA molecule into a mammalian cell, wherein said at least one siRNA molecule has an antisense strand that is at least substantially complementary to a region of non-protein coding target nucleic acid sequence and said at least one siRNA molecule comprises a duplex region of between 25 and 30 base pairs.

2. The method according to claim 1, wherein the non-coding target nucleic acid sequence comprises a promoter.

3. The method according to claim 2, wherein the promoter comprises one or more regulatory sequences.

4. The method according to claim 1, wherein said duplex region comprises between 26 and 29 base pairs.

5. The method of claim 1, further comprising modifying at least one molecule in the mammalian cell.

6. The method of claim 5, wherein the at least one molecule is DNA.

7. The method of claim 5, wherein the at least one molecule is a histone.

8. The method of claim 6, wherein the modifying is by methylation.

9. The method of claim 7, wherein the modifying is by methylation.

10. A method of gene silencing comprising introducing into a mammalian cell at least two siRNA molecules, wherein each of said at least two siRNA molecules is comprised of a sense strand and an antisense strand, each of said antisense strands is at least substantially complementary to a region of non-protein coding nucleic acid target sequence, and within each of said at least two siRNA molecules said sense strand and said antisense strand form a duplex region of between 19 and 30 base pairs.

11. The method according to claim 10, wherein said antisense strand of each of said at least two siRNA molecules is at least substantially complementary to a different region of the same non-protein coding target nucleic acid sequence.

12. The method according to claim 10, wherein the non-protein coding target nucleic acid sequence comprises a promoter.

13. The method according to claim 12, wherein the promoter comprises one or more regulatory sequences.

14. The method according to claim 10, wherein said duplex region comprises between 26 and 29 base pairs.

15. The method according to claim 10, wherein said antisense strands of said at least two siRNA molecules are at least substantially complementary to non-overlapping sequences of said non-protein coding target nucleic acid sequence.

16. The method of claim 10, further comprising modifying at least one molecule in the mammalian cell.

17. The method of claim 16, wherein the at least one molecule is DNA.

18. The method of claim 16, wherein the at least one molecule is a histone.

19. The method of claim 17, wherein the modifying is by methylation.

20. The method of claim 18, wherein the modifying is by methylation.

21. A method of gene silencing comprising introducing at least one siRNA into a mammalian cell, wherein said at least one siRNA molecule is comprised of:
(a) a sense strand;
(b) an antisense strand that is at least substantially complementary to a region of non-protein coding target nucleic acid sequence; and
(c) a nucleus uptake modification located within at least one of said sense strand and said antisense strand.

22. The method according to claim 21, wherein said nucleus uptake modification is comprised of at least one thio modified internucleotide linkage.

23. The method according to claim 21, wherein said nucleus uptake modification is comprised of at least four consecutive thio modified internucleotide linkages.

24. The method according to claim 23, wherein said at least four consecutive thio modified internucleotide linkages are located at a 5′ terminus or a 3′ terminus of at least one strand of said siRNA molecule.

25. The method according to claim 21, wherein the non-protein coding target nucleic acid sequence comprises a promoter.

26. The method according to claim 25, wherein the promoter comprises one or more regulatory sequences.

27. The method according to claim 21, wherein the siRNA molecule comprises a duplex region of between 19 and 29 base pairs.

28. The method of claim 21, further comprising modifying at least one molecule in the mammalian cell.

29. The method of claim 28, wherein the at least one molecule is DNA.

30. The method of claim 28, wherein the at least one molecule is a histone.

31. The method of claim 29, wherein the modifying is by methylation.

32. The method of claim 30, wherein the modifying is by methylation.

33. A method of gene silencing comprising introducing at least two siRNA molecules into a mammalian cell, wherein said at least two siRNA molecules are each comprised of:
(a) a sense strand;
(b) an antisense strand that is at least substantially complementary to a region of non-protein coding target nucleic acid sequence; and
(c) a nucleus uptake modification located within at least one of said sense strand and said antisense strand;
and the antisense strand of each of said at least two siRNA molecules is at least substantially complementary to a different region of the non-protein coding target nucleic acid sequence.

34. The method according to claim 33, wherein said nucleus uptake modification comprises at least four thio modified internucleotide linkages located at a 5′ terminus or a 3′ terminus of at least one of said at least two siRNA molecules.

35. The method according to claim 33, wherein said sense strand and said antisense strand of each of said siRNA molecules forms a duplex of 25-30 base pairs.

36. The method according to claim 33, wherein the non-coding target nucleic acid sequence comprises a promoter.

37. The method according to claim 36, wherein the promoter comprises one or more regulatory sequences.

38. The method according to claim 33, wherein the siRNA molecule comprises a duplex region between 26 and 29 base pairs.

39. The method according to claim 33, wherein said antisense strands of said at least two siRNA molecules are at least substantially complementary to non-overlapping sequences of said non-protein coding target nucleic acid sequence.

40. The method of claim 33, further comprising modifying at least one molecule in the mammalian cell.

41. The method of claim 40, wherein the at least one molecule is DNA.

42. The method of claim 40, wherein the at least one molecule is a histone.

43. The method of claim 41, wherein the modifying is by methylation.

44. The method of claim 42, wherein the modifying is by methylation.


SEQUENCE LISTING (62 sequences)

SEQ ID NO: 1 - DNA, 808 bp, Cytomegalovirus
  promoter (0)...(0): Sequence of promoter region from human cytomegalovirus
tgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctcgcccttaagggcgagcttggtaccgagctcggatccgaaggtaagcctatccctaaccctctcctcggtctcgattctacgcgtaccggtcatcatcaccatc

SEQ ID NO: 2 - DNA, 21 bp, Artificial Sequence
  Combined RNA/DNA, synthetic, RNA with two 2′ deoxythymidines at 3′ end
uguacgggccagauauacgtt

SEQ ID NO: 3 - DNA, 21 bp, Artificial Sequence
  Combined RNA/DNA, synthetic, RNA with two 2′ deoxythymidines at 3′ end
gauauacgcguugacauugtt

SEQ ID NO: 4 - DNA, 21 bp, Artificial Sequence
  Combined RNA/DNA, synthetic, RNA with two 2′ deoxythymidines at 3′ end
uuauugacuaguuauuaautt

SEQ ID NO: 5 - DNA, 21 bp, Artificial Sequence
  Combined RNA/DNA, synthetic, RNA with two 2′ deoxythymidines at 3′ end
caauuacggggucauuagutt

SEQ ID NO: 6 - DNA, 21 bp, Artificial Sequence
  Combined RNA/DNA, synthetic, RNA with two 2′ deoxythymidines at 3′ end
acauaacuuacgguaaaugtt

SEQ ID NO: 7 - DNA, 21 bp, Artificial Sequence
  Combined RNA/DNA, synthetic, RNA with two 2′ deoxythymidines at 3′ end
guauguucccauaguaacgtt

SEQ ID NO: 8 - DNA, 21 bp, Artificial Sequence
  Combined RNA/DNA, synthetic, RNA with two 2′ deoxythymidines at 3′ end
gacuauuuacgguaaacugtt

SEQ ID NO: 9 - DNA, 21 bp, Artificial Sequence
  Combined RNA/DNA, synthetic, RNA with two 2′ deoxythymidines at 3′ end
guaucauaugccaaguacgtt

SEQ ID NO: 10 - DNA, 21 bp, Artificial Sequence
  Combined RNA/DNA, synthetic, RNA with two 2′ deoxythymidines at 3′ end
cauuaugcccaguacaugatt

SEQ ID NO: 11 - DNA, 21 bp, Artificial Sequence
  Combined RNA/DNA, synthetic, RNA with two 2′ deoxythymidines at 3′ end
guacaucuacguauuaguctt

SEQ ID NO: 12 - DNA, 21 bp, Artificial Sequence
  Combined RNA/DNA, synthetic, RNA with two 2′ deoxythymidines at 3′ end
caucaaugggcguggauagtt

SEQ ID NO: 13 - DNA, 21 bp, Artificial Sequence
  Combined RNA/DNA, synthetic, RNA with two 2′ deoxythymidines at 3′ end
uccaaaaugucguaacaactt

SEQ ID NO: 14 - DNA, 21 bp, Artificial Sequence
  Combined RNA/DNA, synthetic, RNA with two 2′ deoxythymidines at 3′ end
guguacggugggaggucuatt

SEQ ID NO: 15 - DNA, 21 bp, Artificial Sequence
  Combined RNA/DNA, synthetic, RNA with two 2′ deoxythymidines at 3′ end
cuagagaacccacugcuuatt

SEQ ID NO: 16 - DNA, 21 bp, Artificial Sequence
  Combined RNA/DNA, synthetic, RNA with two 2′ deoxythymidines at 3′ end
gugauguaugucagagagutt

SEQ ID NO: 17 - DNA, 21 bp, Artificial Sequence
  Combined RNA/DNA, synthetic, RNA with two 2′ deoxythymidines at 3′ end
cauuaugcccaguacaugatt

SEQ ID NO: 18 - DNA, 21 bp, Artificial Sequence
  Combined RNA/DNA, synthetic, RNA with two 2′ deoxythymidines at 3′ end
guacaucuacguauuaguctt

SEQ ID NO: 19 - DNA, 21 bp, Artificial Sequence
  Combined RNA/DNA, synthetic, RNA with two 2′ deoxythymidines at 3′ end
caucaaugggcguggauagtt

SEQ ID NO: 20 - DNA, 21 bp, Artificial Sequence
  Combined RNA/DNA, synthetic, RNA with two 2′ deoxythymidines at 3′ end
uccaaaaugucguaacaactt

SEQ ID NO: 21 - DNA, 27 bp, Artificial Sequence
  Combined RNA/DNA, synthetic, RNA with two 2′ deoxythymidines at 3′ end
gccuggcauuaugcccaguacaugatt

SEQ ID NO: 22 - DNA, 27 bp, Artificial Sequence
  Combined RNA/DNA, synthetic, RNA with two 2′ deoxythymidines at 3′ end
uuggcaguacaucuacguauuaguctt

SEQ ID NO: 23 - DNA, 27 bp, Artificial Sequence
  Combined RNA/DNA, synthetic, RNA with two 2′ deoxythymidines at 3′ end
gcaguacaucaaugggcguggauagtt

SEQ ID NO: 24 - DNA, 27 bp, Artificial Sequence
  Combined RNA/DNA, synthetic, RNA with two 2′ deoxythymidines at 3′ end
ggacuuuccaaaaugucguaacaactt

SEQ ID NO: 25 - DNA, 29 bp, Artificial Sequence
  Combined RNA/DNA, synthetic, RNA with two 2′ deoxythymidines at 3′ end
ccgccuggcauuaugcccaguacaugatt

SEQ ID NO: 26 - DNA, 29 bp, Artificial Sequence
  Combined RNA/DNA, synthetic, RNA with two 2′ deoxythymidines at 3′ end
acuuggcaguacaucuacguauuaguctt

SEQ ID NO: 27 - DNA, 29 bp, Artificial Sequence
  Combined RNA/DNA, synthetic, RNA with two 2′ deoxythymidines at 3′ end
uggcaguacaucaaugggcguggauagtt

SEQ ID NO: 28 - DNA, 29 bp, Artificial Sequence
  Combined RNA/DNA, synthetic, RNA with two 2′ deoxythymidines at 3′ end
cgggacuuuccaaaaugucguaacaactt

SEQ ID NO: 29 - DNA, 21 bp, Artificial Sequence
  Combined RNA/DNA, synthetic, RNA with two 2′ deoxythymidines at 3′ end
tgttcgacgacgccattgatt

SEQ ID NO: 30 - DNA, 27 bp, Artificial Sequence
  Combined RNA/DNA, synthetic, RNA with two 2′ deoxythymidines at 3′ end
cgcguuacauaacuuacgguaaaugtt

SEQ ID NO: 31 - DNA, 27 bp, Artificial Sequence
  Combined RNA/DNA, synthetic, RNA with two 2′ deoxythymidines at 3′ end
uggguggacuauuuacgguaaacugtt

SEQ ID NO: 32 - DNA, 29 bp, Artificial Sequence
  Combined RNA/DNA, synthetic, RNA with two 2′ deoxythymidines at 3′ end
uccgcguuacauaacuuacgguaaaugtt

SEQ ID NO: 33 - DNA, 29 bp, Artificial Sequence
  Combined RNA/DNA, synthetic, RNA with two 2′ deoxythymidines at 3′ end
aauggguggacuauuuacgguaaacugtt

SEQ ID NO: 34 - DNA, 29 bp, Artificial Sequence
  Combined RNA/DNA, synthetic, RNA with two 2′ deoxythymidines at 3′ end
ccgccuggcauuaugcccaguacaugatt

SEQ ID NO: 35 - DNA, 29 bp, Artificial Sequence
  Combined RNA/DNA, synthetic, RNA with two 2′ deoxythymidines at 3′ end
acuuggcaguacaucuacguauuaguctt

SEQ ID NO: 36 - DNA, 29 bp, Artificial Sequence
  Combined RNA/DNA, synthetic, RNA with two 2′ deoxythymidines at 3′ end
uggcaguacaucaaugggcguggauagtt

SEQ ID NO: 37 - DNA, 29 bp, Artificial Sequence
  Combined RNA/DNA, synthetic, RNA with two 2′ deoxythymidines at 3′ end
cgggacuuuccaaaaugucguaacaactt

SEQ ID NO: 38 - DNA, 29 bp, Artificial Sequence
  Combined RNA/DNA, synthetic, RNA with two 2′ deoxythymidines at 3′ end
ccgccuggcauuaugcccaguacaugatt

SEQ ID NO: 39 - DNA, 29 bp, Artificial Sequence
  Combined RNA/DNA, synthetic, RNA with two 2′ deoxythymidines at 3′ end
acuuggcaguacaucuacguauuaguctt

SEQ ID NO: 40 - DNA, 29 bp, Artificial Sequence
  Combined RNA/DNA, synthetic, RNA with two 2′ deoxythymidines at 3′ end
uggcaguacaucaaugggcguggauagtt

SEQ ID NO: 41 - DNA, 29 bp, Artificial Sequence
  Combined RNA/DNA, synthetic, RNA with two 2′ deoxythymidines at 3′ end
cgggacuuuccaaaaugucguaacaactt

SEQ ID NO: 42 - DNA, 21 bp, Artificial Sequence
  Combined RNA/DNA, synthetic, RNA with two 2′ deoxythymidines at 3′ end
gauuauguccgguuauguatt

SEQ ID NO: 43 - DNA, 21 bp, Artificial Sequence
  Combined RNA/DNA, synthetic, RNA with two 2′ deoxythymidines at 3′ end
cugugaauacaaaucacagtt

SEQ ID NO: 44 - DNA, 21 bp, Artificial Sequence
  Combined RNA/DNA, synthetic, RNA with two 2′ deoxythymidines at 3′ end
uacguauuagucaucgcuatt

SEQ ID NO: 45 - DNA, 21 bp, Artificial Sequence
  Combined RNA/DNA, synthetic, RNA with two 2′ deoxythymidines at 3′ end
cguauuagucaucgcuauutt

SEQ ID NO: 46 - DNA, 21 bp, Artificial Sequence
  Combined RNA/DNA, synthetic, RNA with two 2′ deoxythymidines at 3′ end
uauuagucaucgcuauuactt

SEQ ID NO: 47 - DNA, 21 bp, Artificial Sequence
  Combined RNA/DNA, synthetic, RNA with two 2′ deoxythymidines at 3′ end
uuagucaucgcuauuaccatt

SEQ ID NO: 48 - DNA, 21 bp, Artificial Sequence
  Combined RNA/DNA, synthetic, RNA with two 2′ deoxythymidines at 3′ end
agucaucgcuauuaccaugtt

SEQ ID NO: 49 - DNA, 21 bp, Artificial Sequence
  Combined RNA/DNA, synthetic, RNA with two 2′ deoxythymidines at 3′ end
ucaucgcuauuaccauggutt

SEQ ID NO: 50 - DNA, 21 bp, Artificial Sequence
  Combined RNA/DNA, synthetic, RNA with two 2′ deoxythymidines at 3′ end
aucgcuauuaccauggugatt

SEQ ID NO: 51 - DNA, 21 bp, Artificial Sequence
  Combined RNA/DNA, synthetic, RNA with two 2′ deoxythymidines at 3′ end
cgcuauuaccauggugaugtt

SEQ ID NO: 52 - DNA, 21 bp, Artificial Sequence
  Combined RNA/DNA, synthetic, RNA with two 2′ deoxythymidines at 3′ end
cuauuaccauggugaugcgtt

SEQ ID NO: 53 - DNA, 21 bp, Artificial Sequence
  Combined RNA/DNA, synthetic, RNA with two 2′ deoxythymidines at 3′ end
auuaccauggugaugcggutt

SEQ ID NO: 54 - DNA, 21 bp, Artificial Sequence
  Combined RNA/DNA, synthetic, RNA with two 2′ deoxythymidines at 3′ end
uaccauggugaugcgguuutt

SEQ ID NO: 55 - DNA, 21 bp, Artificial Sequence
  Combined RNA/DNA, synthetic, RNA with two 2′ deoxythymidines at 3′ end
ggcaguacaucaaugggcgtt

SEQ ID NO: 56 - DNA, 21 bp, Artificial Sequence
  Combined RNA/DNA, synthetic, RNA with two 2′ deoxythymidines at 3′ end
ggauagcgguuugacucactt

SEQ ID NO: 57 - DNA, 21 bp, Artificial Sequence
  Combined RNA/DNA, synthetic, RNA with two 2′ deoxythymidines at 3′ end
gggauuuccaagucuccactt

SEQ ID NO: 58 - DNA, 21 bp, Artificial Sequence
  Combined RNA/DNA, synthetic, RNA with two 2′ deoxythymidines at 3′ end
ccauugacgucaaugggagtt

SEQ ID NO: 59 - DNA, 21 bp, Artificial Sequence
  Combined RNA/DNA, synthetic, RNA with two 2′ deoxythymidines at 3′ end
uuguuuuggcaccaaaauctt

SEQ ID NO: 60 - DNA, 21 bp, Artificial Sequence
  Combined RNA/DNA, synthetic, RNA with two 2′ deoxythymidines at 3′ end
acgggacuuuccaaaaugutt

SEQ ID NO: 61 - DNA, 21 bp, Artificial Sequence
  Combined RNA/DNA, synthetic, RNA with two 2′ deoxythymidines at 3′ end
guaacaacuccgccccauutt

SEQ ID NO: 62 - DNA, 21 bp, Artificial Sequence
  Combined RNA/DNA, synthetic, RNA with two 2′ deoxythymidines at 3′ end
acgcaaaugggcgguaggctt